(19) United States
(12) Patent Application Publication
(10) Pub. No.: US 2020/0185106 A1
(43) Pub. Date: Jun. 11, 2020

(54) 3-DIMENSIONAL MODEL CREATION USING WHOLE EYE FINITE ELEMENT MODELING OF HUMAN OCULAR STRUCTURES

(71) Applicant: ACE VISION GROUP, INC., Silver Lake, OH (US)

(72) Inventors: AnnMarie Hipsley, Silver Lake, OH (US); Sylvia S. Blemker, Charlottesville, VA (US); Katie R. Knaus, Charlottesville, VA (US)

(21) Appl. No.: 16/702,470

(22) Filed: Dec. 3, 2019

Related U.S. Application Data
(63) Continuation of application No. 15/638,308, filed on Jun. 29, 2017, now abandoned.
(60) Provisional application No. 62/356,457, filed on Jun. 29, 2016.

(51) Int. Cl. G16H 50/50; G06T 17/20; A61B 3/00; G06F 30/23

(52) U.S. Cl. CPC G16H 50/50; G06F 30/23; A61B 3/00; G06T 17/20

(57) ABSTRACT

Disclosed are systems, devices and methods for a modeling of ocular structures involved in ocular accommodation and use of a multi-component Finite Element Model (FEM).

### CROSS REFERENCE TO RELATED APPLICATIONS

This application is a continuation of U.S. patent application Ser. No. 15/638,308, filed Jun. 29, 2017, which claims priority pursuant to 35 U.S.C. § 119(e) to U.S. Provisional Patent Application No. 62/356,457, filed Jun. 29, 2016, the disclosures of both which are hereby incorporated by reference in their entireties.

This application is related to the subject matter disclosed in U.S. Appl. No. 61/798,379, filed Mar. 15, 2013; U.S. Appl. No. 60/662,026, filed Mar. 15, 2005; U.S. application Ser. No. 11/376,969, filed Mar. 15, 2006; U.S. Appl. No. 60/842,270, filed Sep. 5, 2006; U.S. Appl. No. 60/865,314, filed Nov. 10, 2006; U.S. Appl. No. 60/857,821, filed Nov. 10, 2006; U.S. application Ser. No. 11/850,407, filed Sep. 5, 2007; U.S. application Ser. No. 11/938,489, filed Nov. 12, 2007; U.S. application Ser. No. 12/958,037, filed Dec. 1, 2010; U.S. application Ser. No. 13/342,441, filed Jan. 3, 2012; U.S. application Ser. No. 14/526,426, filed Oct. 28, 2014; U.S. application Ser. No. 14/861,142, filed Sep. 22, 2015; U.S. application Ser. No. 11/850,407, filed Sep. 5, 2007; U.S. application Ser. No. 14/213,492, filed Mar. 14, 2014, the entire contents and disclosures of which are hereby incorporated by reference.

### FIELD OF THE INVENTION

The subject matter described herein relates generally to systems, methods and devices for creating 3-dimensional models of complete ocular FEM of human ocular accommodation that can be used in simulating the biomechanical properties of connective tissue structure and function. Additionally, the subject matter described herein relates to systems, methods and devices for modeling connective tissue changes by analyzing and experimentation on the underlying biomechanical properties of the connective tissue.

### BACKGROUND OF THE INVENTION

As people age, they develop presbyopia and lose accommodative ability, leaving people over the age of 50 with an almost complete lack of focusing ability for near vision. Although scientists have studied accommodation for centuries the functional mechanism is not well understood. Most presbyopia research has focused on property changes of the aging lens without examining the accommodative mechanism as a whole, basically ignoring the complicated role of the ciliary muscle. Without understanding the interactions of the muscle, lens, and other structures that alter the eye's optic power, treatments for presbyopia that effectively restore this ability cannot be successfully developed. This lack of understanding is also in part due to the limited data, especially in vivo or dynamic, of healthy human eyes; most current measurement techniques require isolating or disturbing some portion of the accommodative system and are limited to cadavers or monkey models. These data provide a disjointed comprehension of the accommodative mechanism and the implications of age-related changes to eye structure.

Currently Goldberg's Postulate incorporates all elements of the zonular apparatus into the phenomenon of accommodation. Biometry has shown lens thickness increases and the anterior chamber depth decreases upon contraction of the ciliary muscles, the lens capsule steepens, as the posterior-lens surface moves backwards. There is a decrease in the distance from scleral spur to the ora serrata, the Nasal sclera compresses inward and the Choroid also stretches forward.

A computational model is critical to understanding how the complex movements of the ciliary muscle drive the lens changes necessary for accommodation, and to understand how age-related changes lead to presbyopia. Most previous models focused solely on the actions of lens and zonules, simplifying ciliary movement to a single displacement, and simulating the transition from the accommodated state where the lens is un-stretched but the muscle is contracted, to the unaccommodated state where the muscle is at rest and the lens is stretched. This method depends on a simplified arrangement of the zonule attachments and also ignores the complex behaviors of the ciliary muscle, whose movements are constrained by its attachments to the sclera and choroid. The goal of this study was to develop a multi-component finite element (FE) model of the accommodative mechanism that includes the ciliary muscle, lens, zonules, sclera, and choroid, to characterize the role of complex ciliary muscle action in producing the lens changes required for accommodative function.

Development of accurate computational models is critical in order to advance scientific understanding regarding how ocular ciliary muscle movements result in changes during accommodative processes and their results on an associated ocular lens. Particularly, these models can help to understand how age-related changes in ocular structures lead to age-related dysfunctions and pathophysiology such as presbyopia, age-related glaucoma, age related macular degeneration, cataract formation and others. Accommodation mechanisms are highly complex and difficult to analyze, especially those of the ciliary body (muscles) which are under emphasized and grossly overlooked and not well characterized to date.

Most prior art accommodation models focus solely on the actions of lenses and zonules in isolation of extralenticular structures and whole eye biomechanics, and thus, oversimplify ciliary movement as a single muscular displacement. In particular, the emphasis for ocular accommodation to date has typically been focused on identifying and creating changes in ocular lens properties, while not addressing underlying ciliary muscle operations. These models simulate the transition from an accommodated state, where a lens is un-stretched but the associated ciliary muscle is contracted, to an unaccommodated state, where the ciliary muscle is at rest and the lens is stretched. Unfortunately, these models depend on a simplified arrangement of zonule attachments and ignore or otherwise neglect the uniquely complex behaviors of the ciliary muscle, whose movements are constrained by attachments to the ocular sclera and choroid structures.

Due to the simplification of the ciliary muscle behaviors as applied in these prior art models, attempts to apply pre-tensioning of zonules prior to ciliary muscle contraction have not been successful. This has led not only to a gap in the understanding of the accommodation mechanism but also to a lack of effective treatment in restoring the accommodative functions that the conditions created by presbyopia and other age-related eye afflictions, including proper aqueous flow hydrodynamics and normal organ function to name a few.

Also contributing to the lack of effective treatment for deteriorated accommodative function is the fact that there is an overall scarcity of data with respect to the functioning accommodative mechanisms for healthy human eyes, especially in vivo or dynamic data. Since accommodative functioning is difficult to measure because of the delicate nature of the human eye, most current measurement techniques have relied on data gathered from experimentation on the ocular systems of human cadavers and other primates. Gathering this data usually requires isolating or disturbing at least a portion of the accommodative ocular system, making procedures difficult and dangerous for live human test subjects.

As a result of insufficient data regarding the accommodative ocular system, its underlying mechanisms and the related problem of incomplete modeling, analysis of existing data provides a disjointed and incomplete understanding of ocular accommodation in humans and any implications resulting from age-related changes to ocular structures.

Various examples of prior art creating meshed finite element models include U.S. Patent Pub. No. 2007/0027667, U.S. Pat. Nos. 8,346,518, 7,798,641, and 7,096,166. U.S. Patent Publ. No. 2007/0027667 in particular serves as a general example how to specify “Computational Model of Human ocular accommodative biomechanics in young and old adults.” These prior art applications generally do not perform simulations on an entire eye, particularly an entire human eye, and do not include simulations, analyzers, artificial intelligence and machine learning and other important concepts and aspects disclosed herein.

It is therefore desirable to provide improved systems, devices and methods for a multi-component Finite Element Model (FEM) of an ocular accommodative mechanism that includes ocular structures including the ciliary muscle, lens, zonules, sclera, and choroid, in order to characterize the role of complex ciliary muscle action in producing ocular lens changes required for accommodative function between young and presbyopic adults. This can be accomplished through improved modeling techniques in order to gain a better understanding of how ciliary muscle function modification may lead to improved medical treatments, since most scientific research to date has been focused on the change in lens properties instead of muscle action.

### SUMMARY OF THE INVENTION

Disclosed are systems, devices, and methods for creating a multi-component Finite Element Model (FEM) of ocular structures involved in ocular accommodation. Developing a computational model can be critical to understanding how the complex movements of the ciliary muscle drive the lens changes necessary for accommodation, and to understand how age-related changes lead to presbyopia. Most prior models focused solely on the actions of lens and zonules, simplifying ciliary movement to a single displacement. In particular, these models function by simulating the transition from the accommodated state where the lens is un-stretched but the muscle is contracted to the unaccommodated state where the muscle is at rest and the lens is stretched. As such, the disclosed developments of multi-component FEMs of the accommodative mechanism that include the ciliary muscle, lens, zonules, sclera, and choroid, to characterize the role of complex ciliary muscle action in producing the lens changes required for accommodative function.

The principles and concepts disclosed herein can be used to create and facilitate visualization of accommodation structures. They can also be used to measure, evaluate and predict central optical power. Additionally, they can be used to simulate age specific whole or partial eye structures, functions, and biomechanics. Further, they can be used to independently simulate the ciliary muscle and its components, extra-lenticular, and lenticular movements, and functions on the lens. Also, individual simulations of anatomical structures and fibers can be performed that can reveal some biomechanical relationships that have otherwise been unknown or otherwise undefined and under-researched.

Numerical simulation of the patient's eye can be created using 3D FEM meshing to accomplish methods such as adding a “pre-stretch” lens positioning in coding and manipulations of software, as executed by a computer processor. Similarly, methods of intricate meshing of zonular and other structures, methods of importing dynamic imaging into models for the purposes of modelling accommodation and accommodative movements including, but not limited to, simulation of central optical power and changes in the crystalline lens can be accomplished using computer-based computations. Additionally, methods and software manipulation executed by a processor can be capable of performing numerical simulation of zonular apparatus movements, forces and impact on Central Optical Power (COP).

Systems, methods and devices disclosed herein can be used to perform other functions as well, such as those pertaining to modelling other structures of the eye, such as the back of the eye, including: lamina cribrosa, Ocular Nerve Head and others, related to ocular structures and functions. For example, regarding the posterior globe: new insights and understanding of the lamina cribrosa are possible, as are insights into the complex structure of the peripapillary sclera, and attachments of the choroid using complex math for solving elastic and viscoelastic equations and simulations may provide additional benefits.

In particular, the structural behavior of the whole eye, which is governed by the material properties, physics, biomechanics and behavior of the optics under various conditions and can be modeled as a 3D computer mathematical simulation for later use in predicting future ocular conditions. The proposed simulations in creating computational models and the effects of surgical procedures implemented using them can be based on a number of important underlying simplified assumptions regarding the mechanical properties and structure of the ocular tissues at the ultrastructure level. As such, more accurate modeling is desirable for diagnostic, surgical planning, intraoperative surgical adjustment, and virtual surgical simulation.

Modeling of the eye can answer various questions about the eye. Some examples include: how does regional restoration of sclera stiffness improve ciliary deformation in accommodation? Do certain zones or combinations of zones have a greater effect? Does regional restoration of sclera attachment tightness (in addition to stiffness) augment improvements to ciliary deformation in accommodation? How do the treatment parameters relate to the change in scleral stiffness in the treated regions? How does regional restoration with different treatments (therefore different sclera stiffness's) improve ciliary deformation in accommodation?

Methods disclosed herein include: adding a “pre-stretch” lens positioning whether it be code, manipulations of software and the like; intricate meshing of zonular and other structures; importing dynamic imaging into the model for the purposes of modelling accommodation and accommodative movements including but not limited to simulation of central optical power changes in the crystalline lens; software manipulation capable of performing numerical simulation of zonular apparatus movements, forces and impact on COP; modelling the back of the eye: lamina cribrosa, Ocular Nerve Head, and others; posterior globe code for understanding lamina cribrosa; complex structuring of the peripapillary sclera, attachments of the choroid for example; complex math for solving elastic and viscoelastic equations and simulations; zonular reconstruction with relational lens effects by pretension modification of software code and mathematical assumptions along with simulations; simulations or presentations of imaging and math code to display functional relationships; and others.

Thus, simulation models of ocular structures, such as those used in ocular accommodation can be executed and repeated with different versions of an ocular mesh, along with various pluralities of external and internal manipulation of anatomical and geometrical or quasi-physical components.

### BRIEF DESCRIPTION OF THE DRAWING(S)

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely. Illustrated in the accompanying drawing(s) is at least one of the best mode embodiments of the present invention.

FIG. 1A shows an example embodiment of an anatomical diagram of an eye cross section with a reference key.

FIGS. 1B-1C show an example embodiment of a cross-section of an eye diagram and illustrating changes in structural components of an eye for distance and near vision respectively.

FIG. 1D shows an example embodiment diagram of how an unaccommodated eye focuses an image through a lens.

FIG. 1E shows an example embodiment diagram of how an accommodated eye focuses an image through a lens.

FIG. 1F shows an example embodiment of an ocular structure diagram showing ocular structures from a view of the back of a human eye.

FIG. 1G shows an example embodiment of an ocular structure diagram showing ocular structures from a view of the front or anterior view of a human eye.

FIGS. 2A-2B shows an example embodiment of an unaccommodated eye cross sectional image and an accommodated eye cross sectional image, respectively.

FIG. 3A shows an example embodiment of a cross sectional diagram of an eye based on model structures from existing imaging literature.

FIG. 3B shows an example embodiment of a Scanning Electron Microscopy image of Zonular fibers, and nodal attachments as well as pathway of the zonular proximal and distal insertion zones of an eye, based on model structures from existing imaging literature.

FIG. 3C shows an example embodiment of a Scanning Electron Microscopy image of Zonular fibers and relationship to the lens and the Vitreous membrane of an eye based on model structures from existing imaging literature.

FIG. 3D shows an example embodiment diagram of a ciliary body. In general, ciliary body includes ciliary muscle.

FIG. 3E shows an example embodiment image of a cross-section of the anterior segment of the eye showing the accommodation apparatus and related anatomy as well as the whole eye shell and cornea based on model structures from existing imaging literature.

FIG. 3F shows an example embodiment of an ultrasound biometry image of a cross-section of the anterior segment showing the accommodation apparatus, specifically of the relationship of the ciliary process & ciliary body to the posterior vitreal zonule or pars plana, lens, and cornea of an eye, based on model structures from existing imaging literature.

FIG. 3G shows an example embodiment of a Scanning Electron Microscopy image of the relationship between the vitreous membrane, the posterior vitreous zonule insertion and the other zonular structures of an eye based on model structures from existing imaging literature.

FIG. 4A shows an example embodiment flow diagram of a process of developing new ideas for improved treatments.

FIG. 4B shows an example embodiment of a cross sectional diagram for a two-dimensional model design for an eye with enlarged inset to show enhanced detail.

FIG. 4C shows an example embodiment diagram of a three-dimensional model of an eye from a perspective view, side view, and side cross-sectional view.

FIG. 4D shows an example embodiment diagram of a three-dimensional meshing model of an eye from a bottom perspective view, top perspective view, and side cross-sectional view.

FIG. 5A shows an example embodiment of a two-dimensional cross-sectional diagram for a two-dimensional model design for an eye showing measurements of unaccommodated ocular structures.

FIG. 5B shows an example embodiment of a prior art cross sectional image for a two-dimensional model design for an eye showing measurements of unaccommodated ocular structures.

FIG. 5C shows an example embodiment diagram of prior art cross sectional images for a two-dimensional resting human eye showing measurements of unaccommodated ocular structures.

FIG. 6A shows an example embodiment of a cross sectional diagram for a two-dimensional model design for an eye showing variables of accommodated ocular structures.

FIG. 6B shows an example embodiment of a cross sectional diagram for a two-dimensional model design for an eye showing dimensions of accommodated ocular structures.

FIG. 7A shows an example embodiment of a cross-sectional 3-dimensional model structure diagram showing a shaded sclera of an eye.

FIG. 7B shows an example embodiment of a cross-sectional 3-dimensional model structure diagram showing a shaded vitreous membrane of an eye.

FIG. 7C shows an example embodiment of a cross-sectional 3-dimensional model structure diagram showing a shaded lens of an eye.

FIG. 7D shows an example embodiment of a cross-sectional 3-dimensional model structure diagram showing a choroid of an eye.

FIG. 7E shows an example embodiment of a cross-sectional 3-dimensional model structure diagram showing a cornea of an eye.

FIG. 7F shows an example embodiment of a cross-sectional 3-dimensional model structure diagram showing a capsule, cortex, and nucleus of an ocular lens.

FIG. 7G shows an example embodiment of a cross-sectional 3-dimensional model structure diagram showing various ocular structures of an eye.

FIG. 7H shows an example embodiment of a cross-sectional 3-dimensional model structure diagram showing a shaded ciliary muscle of an eye.

FIG. 7I shows an example embodiment of a cross-sectional 3-dimensional model structure diagram showing shaded zonules of an eye.

FIG. 7J shows an example embodiment of a cross-sectional 3-dimensional model structure diagram showing a sclera of an eye.

FIG. 7K shows an example embodiment of a cross-sectional 3-dimensional model structure diagram showing a shaded lens of an eye, including capsule, cortex, and nucleus.

FIG. 7L shows an example embodiment of a cross-sectional 3-dimensional model structure diagram showing a shaded choroid, vitreous membrane, and cornea.

FIG. 8 shows an example embodiment of a cross-sectional 3-dimensional model structure diagram showing a zonules model of an eye with enlarged inset to show enhanced detail.

FIG. 9A shows an example embodiment of a prior art diagram of ciliary fibers of an eye.

FIG. 9B shows an example embodiment of an accommodated eye diagram. As the schematic diagram of the eye is shown, major structures involved in accommodation include: a corneo-scleral shell, a crystalline lens, a ciliary body containing ciliary muscles, and the zonular fibers connecting the ciliary body to the crystalline lens.

FIG. 9C shows an example embodiment of a disaccomodated eye. Here, cornea is coupled with sclera.

FIG. 9D shows an example embodiment of a cross-sectional 3-dimensional model structure diagram showing an integrated composite ciliary fiber model of an eye including an exploded view with separate longitudinal layer model, radial layer model, and circular layer model.

FIG. 10A shows an example embodiment of a cross-sectional 3-dimensional model structure diagram of an eye with enlarged inset to show a meshing model.

FIG. 10B shows an example embodiment diagram of a meshing process.

FIG. 10C shows an example embodiment chart of material parameters of ocular structures.

FIG. 10D shows an example embodiment chart of various formulas governing transversely isotropic materials.

FIG. 10E shows an example embodiment chart of parameters for ciliary muscle and zonules.

FIG. 10F shows an example embodiment of a user interface screen for modifying various parameters during modeling.

FIG. 10G shows an example embodiment chart of strain energy density equations for ciliary muscle and zonules. These can be physically based strain invariants.

FIG. 10H shows an example embodiment chart of dilational strain equations.

FIG. 10I shows an example embodiment chart of along-fiber shear equations and diagram.

FIG. 10J shows an example embodiment chart of cross-fiber shear equations and diagrams.

FIG. 10K shows an example embodiment chart of along-fiber stretch equations and diagrams for ciliary muscles, including activation versus time and force versus fiber length.

FIG. 10L shows an example embodiment chart of along-fiber stretch equations and diagrams for zonules, including pretension versus time and stress versus fiber length.

FIG. 11A shows an example embodiment perspective view of a cross-sectional three-dimensional model structure diagram of an eye.

FIG. 11B shows an example embodiment perspective view of a cross-sectional three-dimensional model structure diagram of an eye.

FIG. 11C shows an example embodiment side view of a cross-sectional three-dimensional model structure diagram of an eye.

FIGS. 12A-12B show an example embodiment of a cross-sectional three-dimensional model structure diagram with upper and lower boundaries of an eye, respectively.

FIGS. 12C-12D shows an example embodiment of a cross-sectional three-dimensional quarter model structure diagram of an eye with radial symmetry and having a right and left boundary, respectively.

FIG. 12E shows an example embodiment of a user interface screen for modifying various parameters during modeling.

FIG. 13A shows an example embodiment of a cross-sectional 7T MRI image of a small animal eye showing anatomy and the relationship of Sagittal macro and micro structures.

FIG. 13B shows an example embodiment of a close-up cross-sectional 7T MRI image of a small animal eye SE showing whole eye anatomy and the relationship of Sagittal macro and micro structures.

FIG. 13C shows an example embodiment of a cross-sectional 7T MRI image of a small animal eye GE showing a whole eye ciliary body.

FIG. 14A shows an example embodiment of a simulation flowchart showing an initial model at rest undergoing zonule pre-tensioning to become an unaccommodated model and ciliary muscle contraction to become an accommodated model.

FIG. 14B shows an example embodiment of an unaccommodated eye diagram.

FIG. 14C shows an example embodiment of an accommodated eye diagram.

FIG. 14D shows example embodiment diagram calling out various components of the anatomy of an eye.

FIG. 14E shows an example embodiment diagram of an accommodation simulation process.

FIG. 14F shows an example embodiment diagram showing tension of zonules versus simulation time and ciliary muscle activation versus time.

FIG. 14G shows an example embodiment user interface diagram of an informational display during simulation screen.

FIG. 15A shows an example embodiment of a diagram including a cross-sectional diagram of an eye with expanded lens image, expanded ciliary muscle for confocal image, and expanded choroid image.

FIG. 15B shows an example embodiment diagram including a cross-sectional diagram of an eye including a ciliary muscle and processes image.

FIGS. 16A-16C are cross-sectional confocal images, respectively, showing ciliary fiber structures and fiber orientations.

FIG. 16D shows an example embodiment diagram of three parts of the ciliary muscle structure. The ciliary body contains the ciliary muscle.

FIGS. 16E-16F show example embodiment diagrams of a corneo-scleral shell with a ciliary body.

FIG. 16G shows an example embodiment diagram of changes in the eye between an unaccommodated eye in central section for distance vision and accommodated eye in right section for near vision.

FIGS. 16H-16I show example embodiments of a disaccomodated eye ciliary muscle diagram from a top view and accommodated eye ciliary muscle diagram from a top view, respectively.

FIGS. 16J-16K show example embodiments of a computer model of ciliary muscles of an eye from a top view and side cross-sectional view with inset respectively.

FIGS. 16L-16N show example embodiment diagrams of longitudinal fibers, radial fibers, and circular fibers, individually modeled and operable to be show simulations of their function during the accommodative process.

FIG. 160 shows an example embodiment diagram of normalized force versus relative length of ciliary muscle.

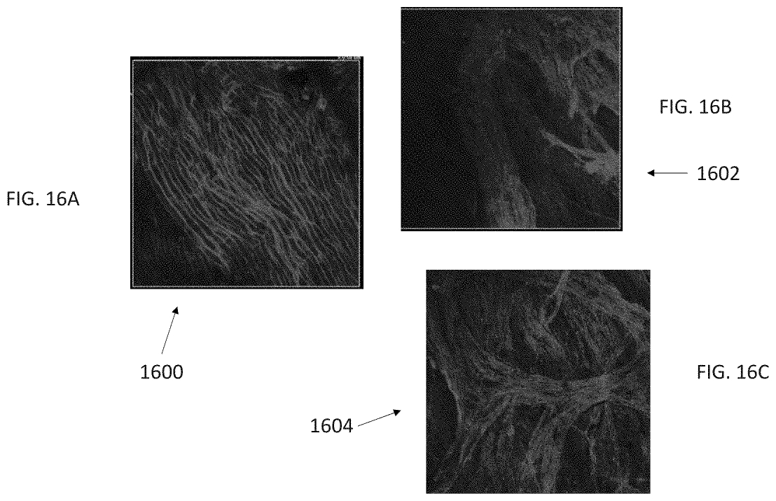

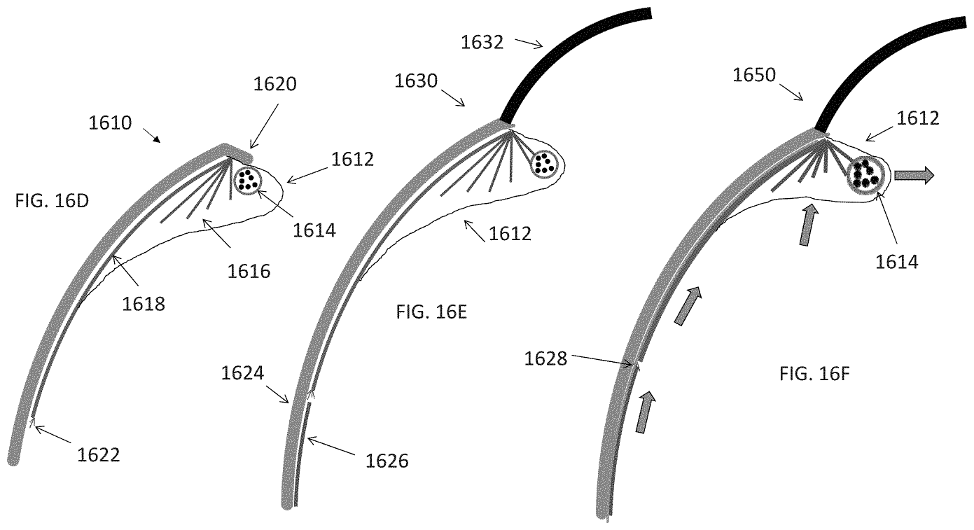

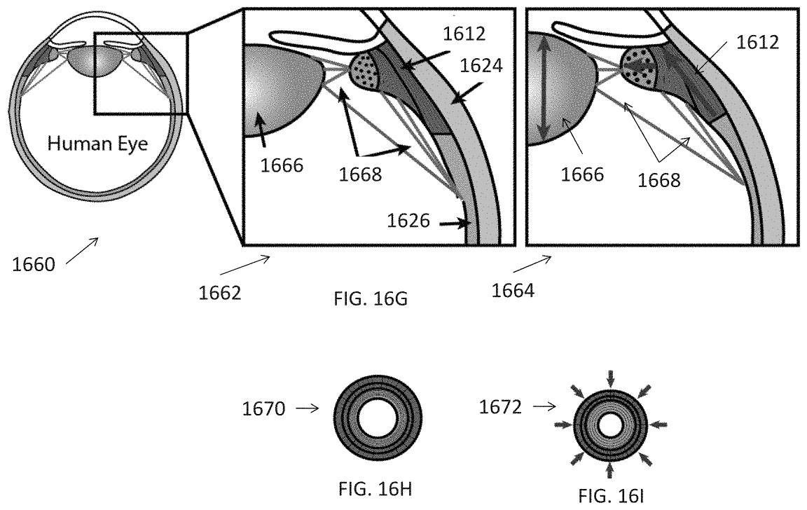

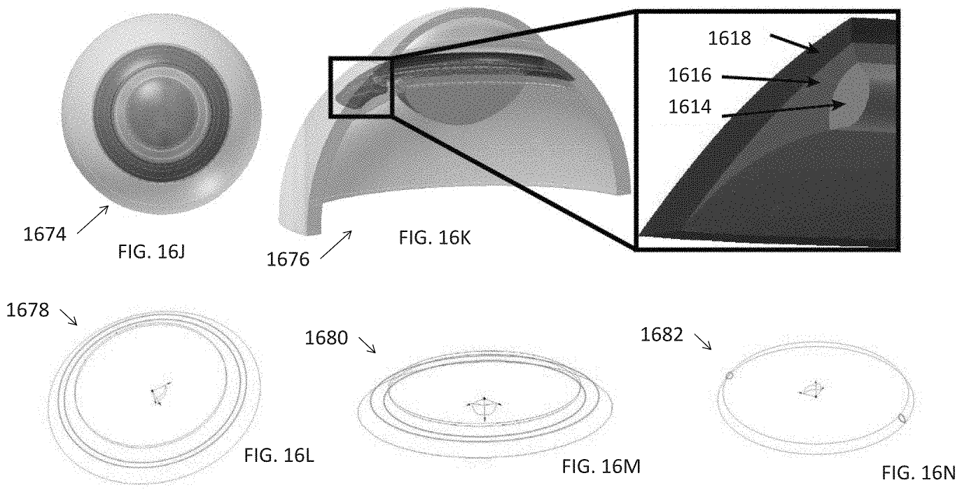

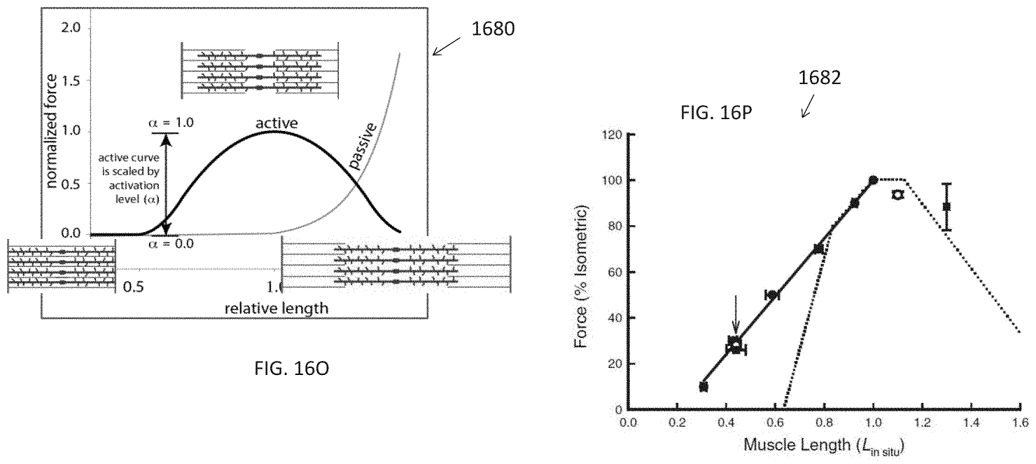

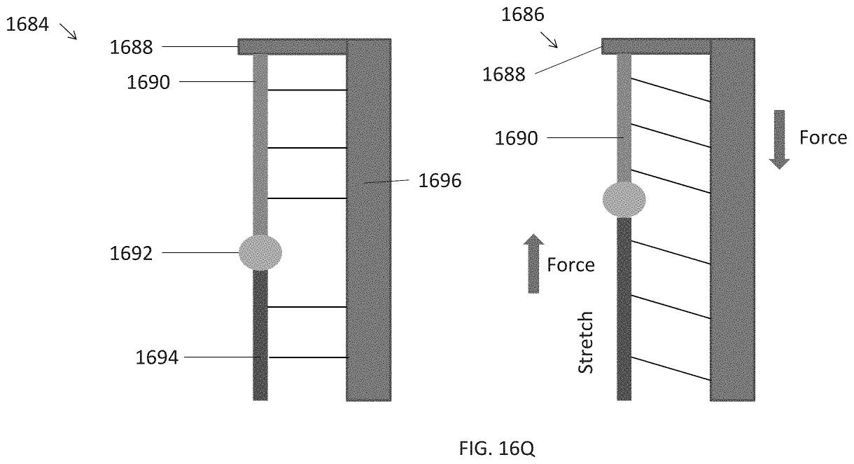

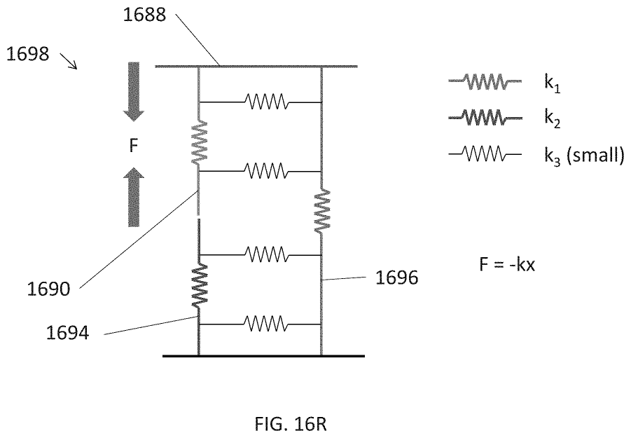

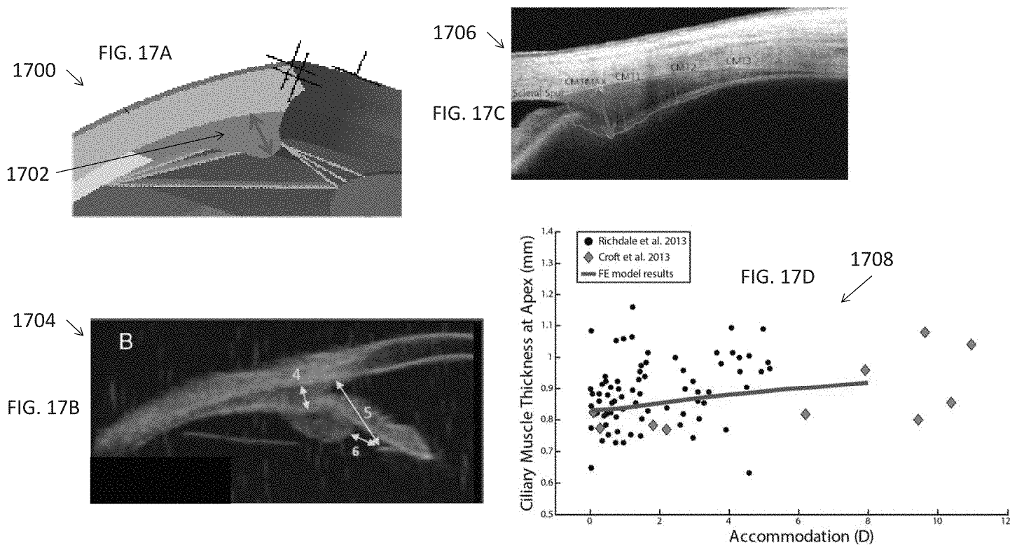

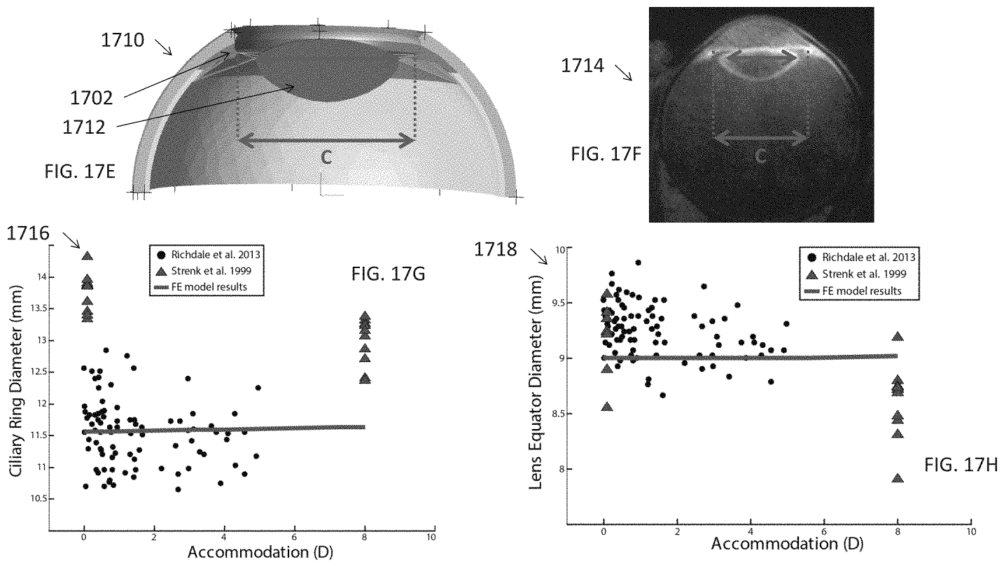

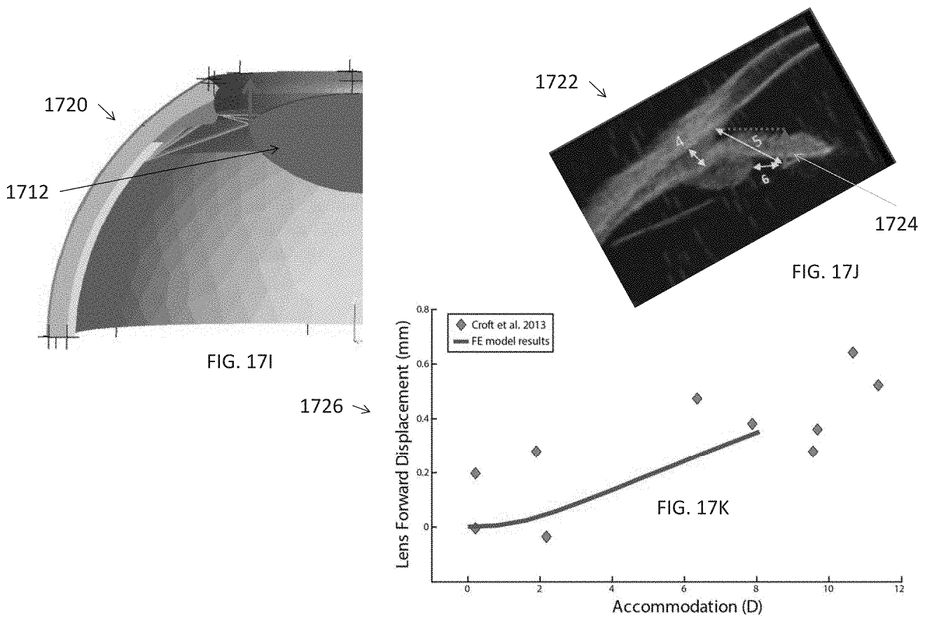

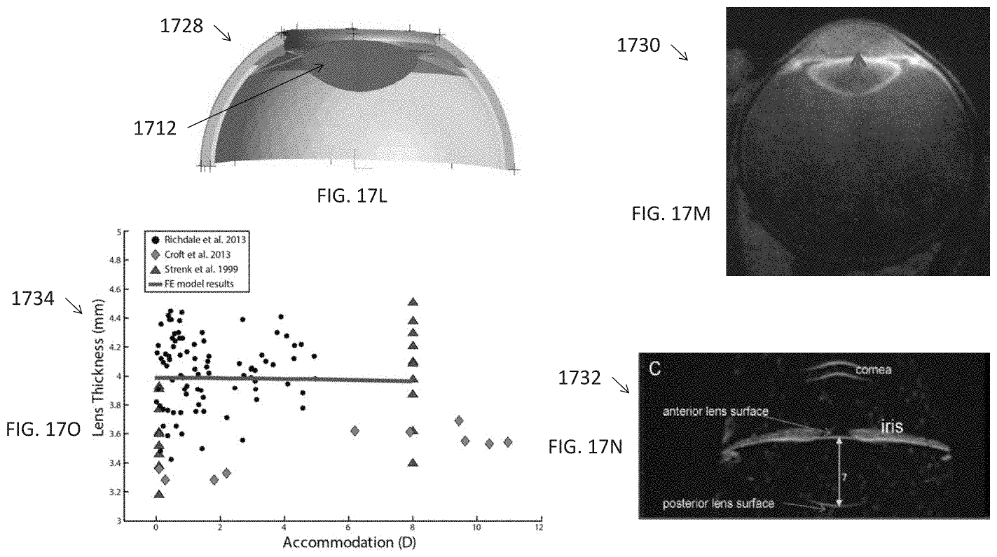

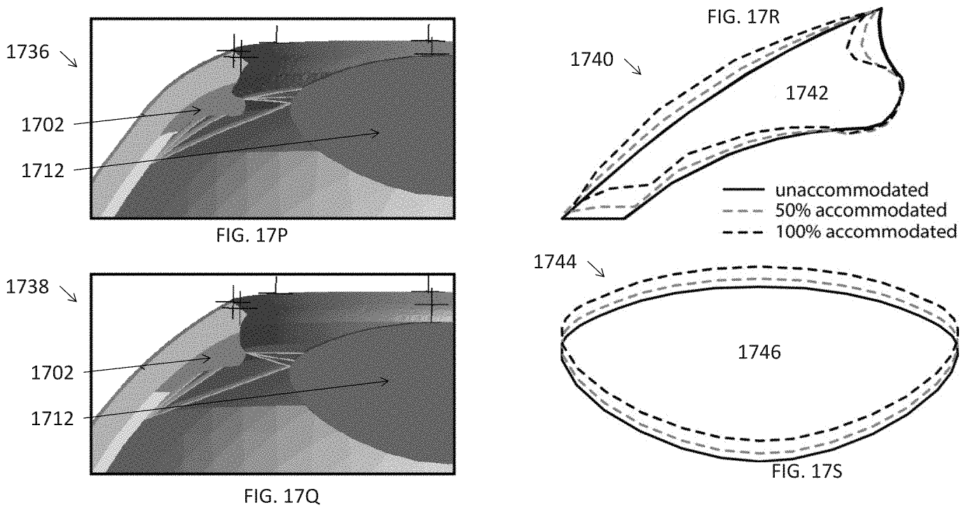

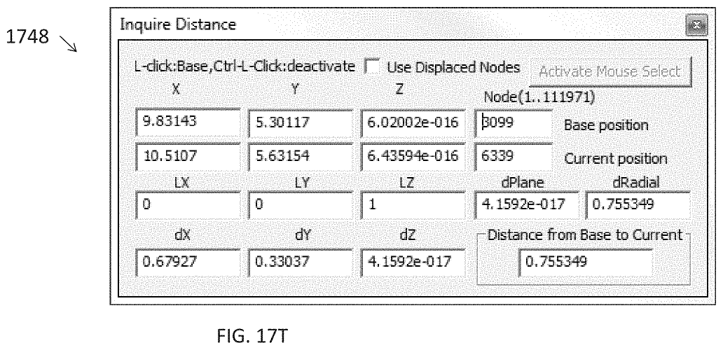

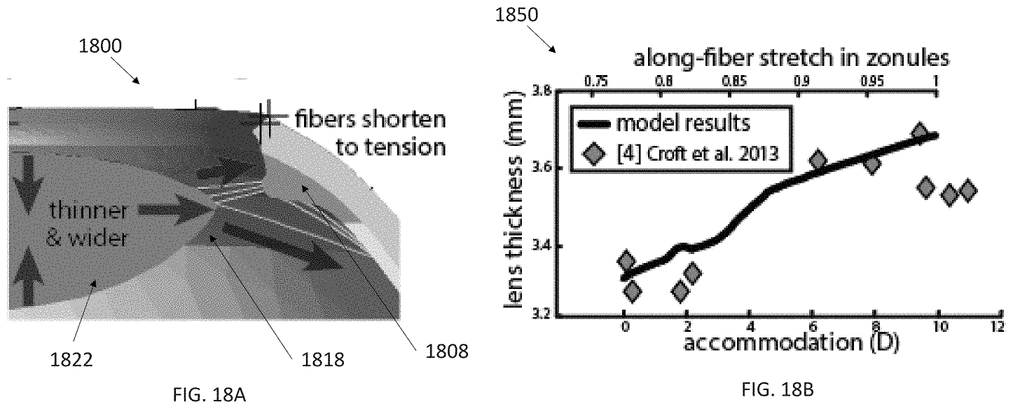

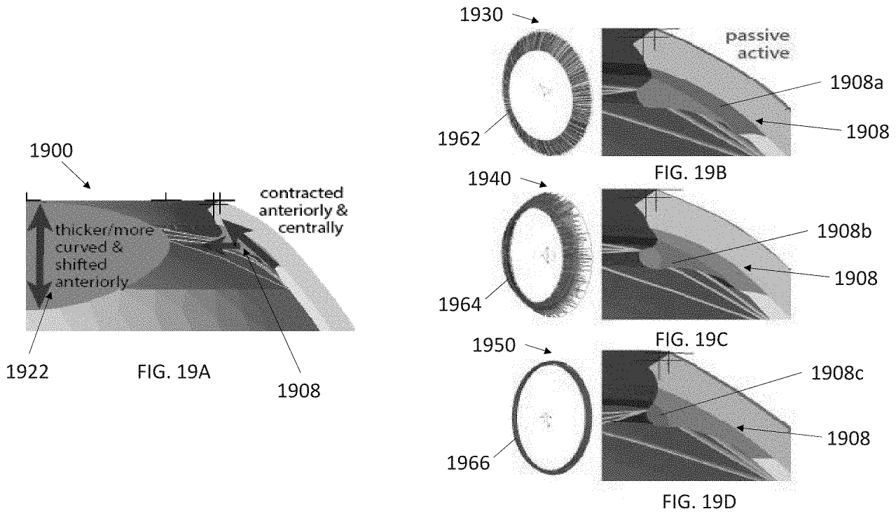

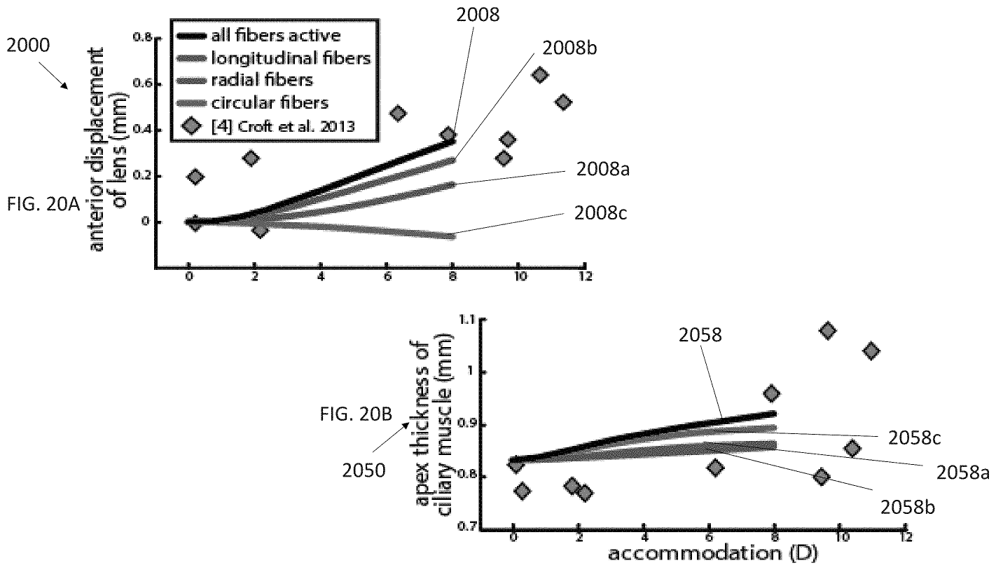

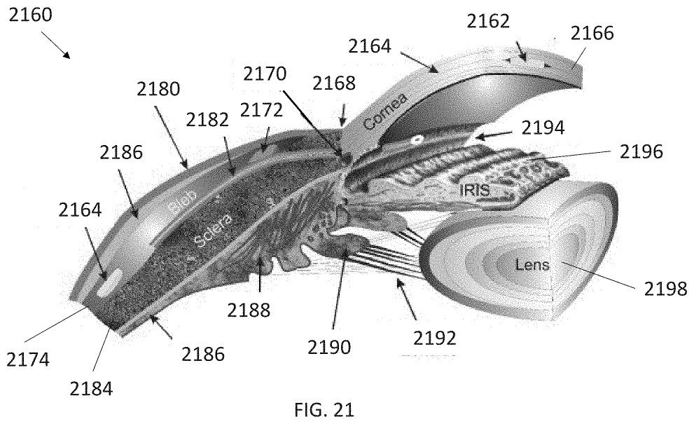

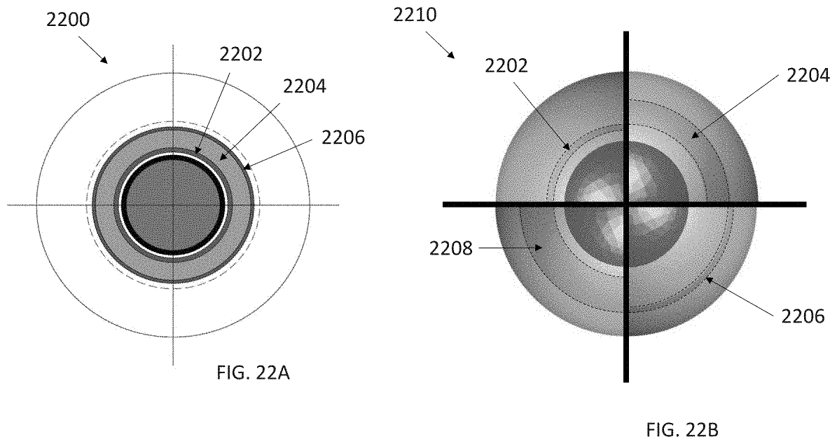

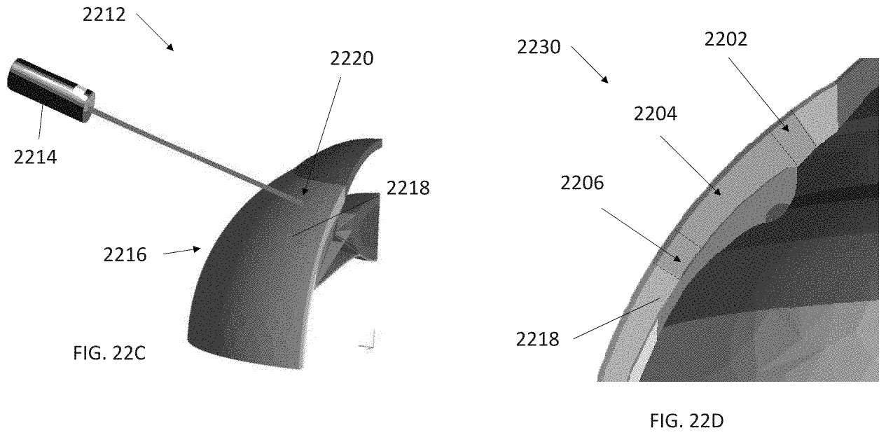

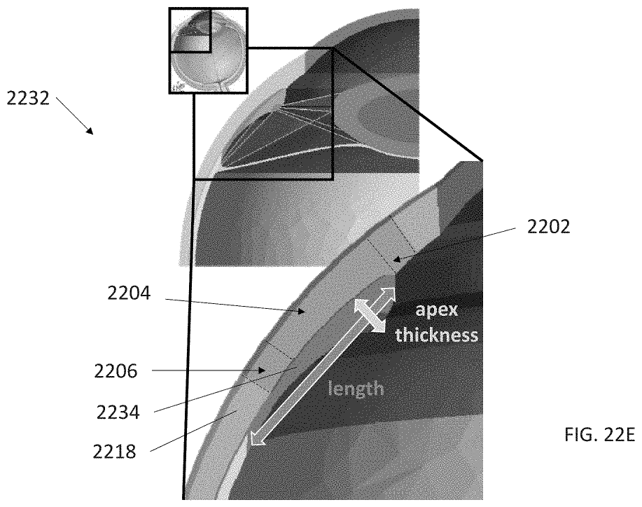

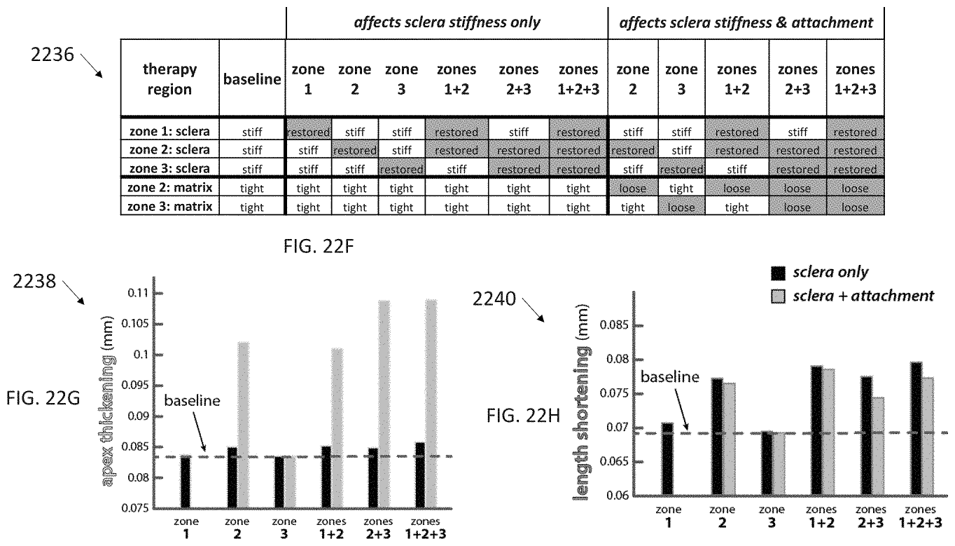

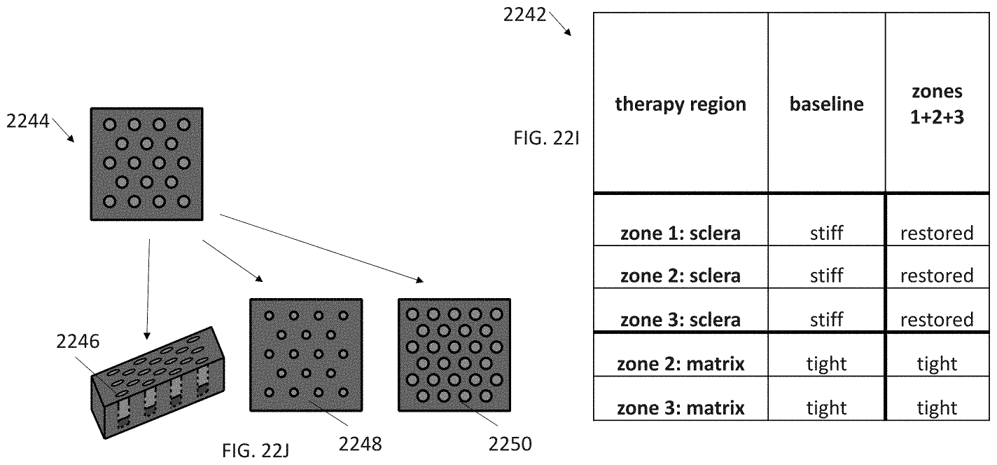

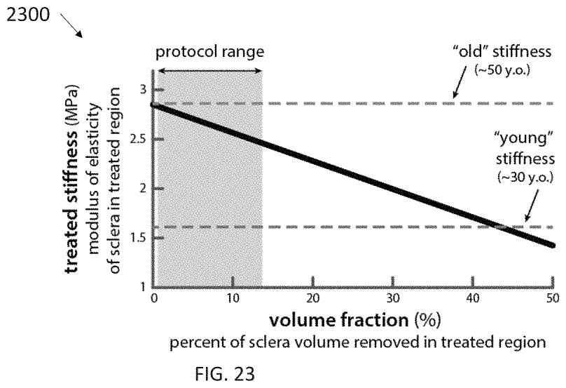

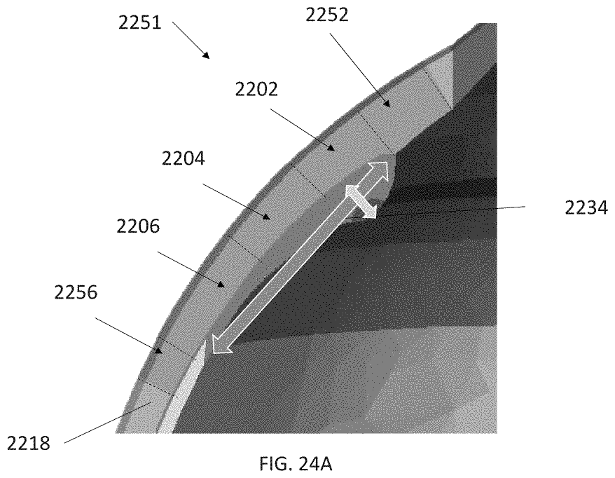

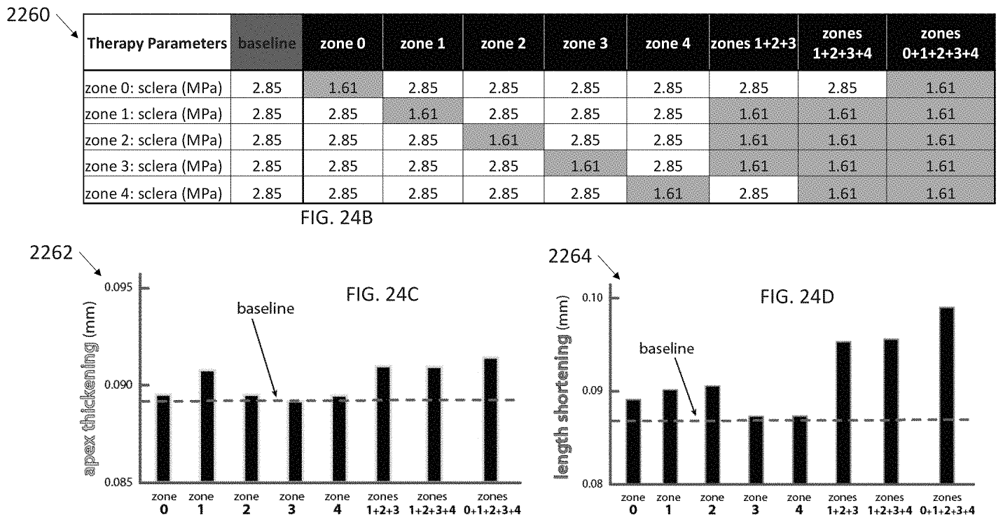

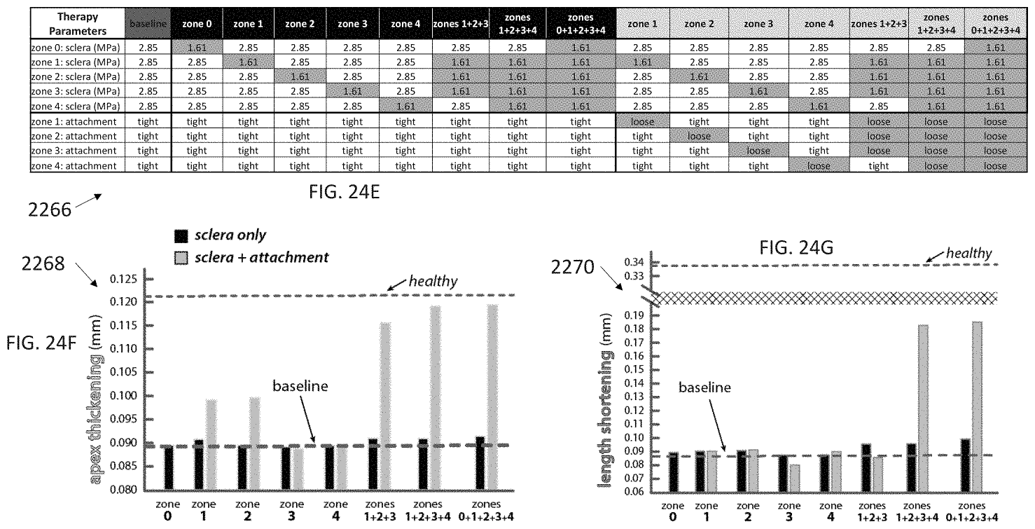

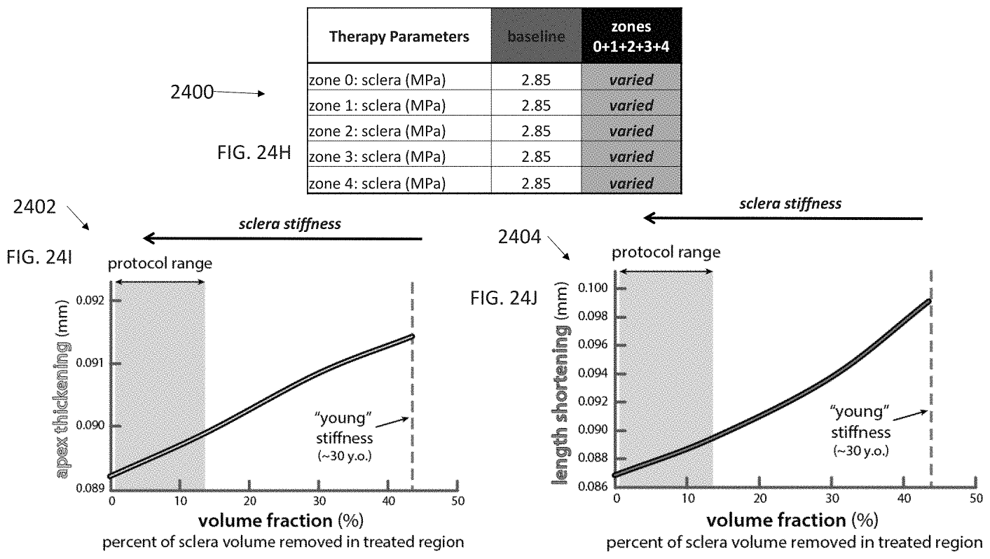

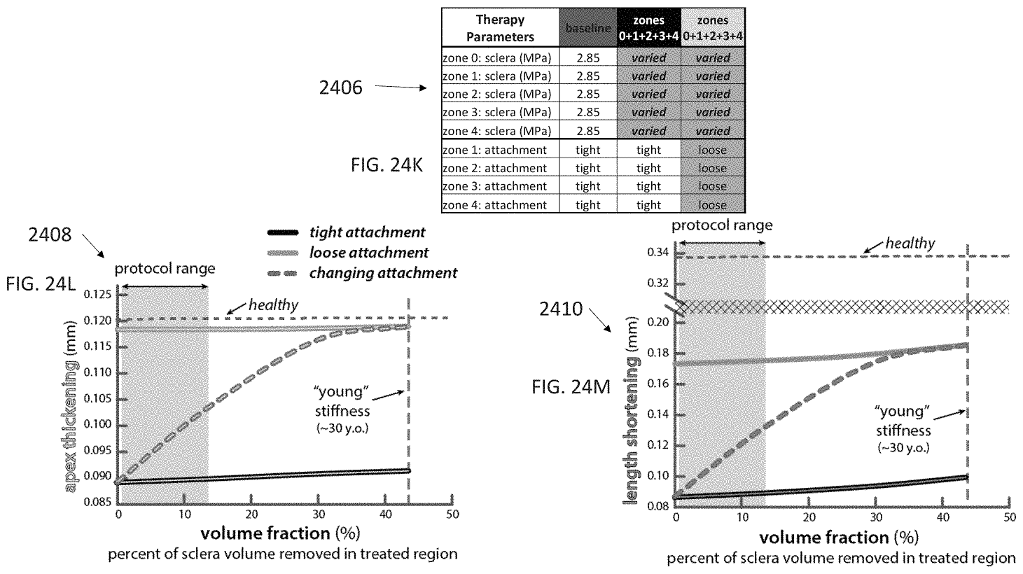

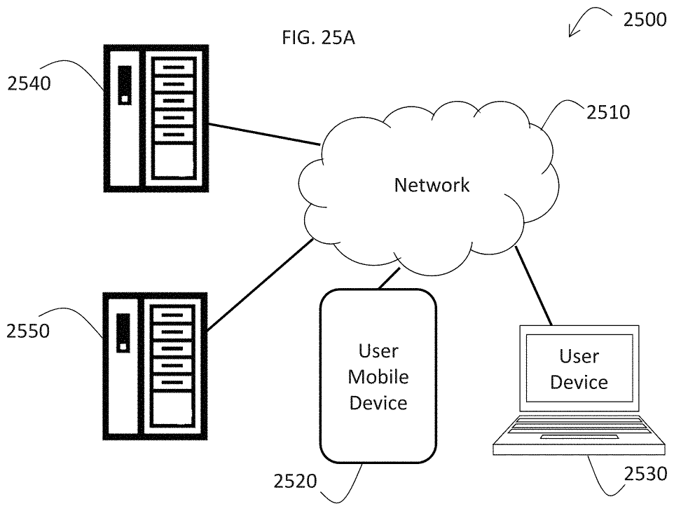

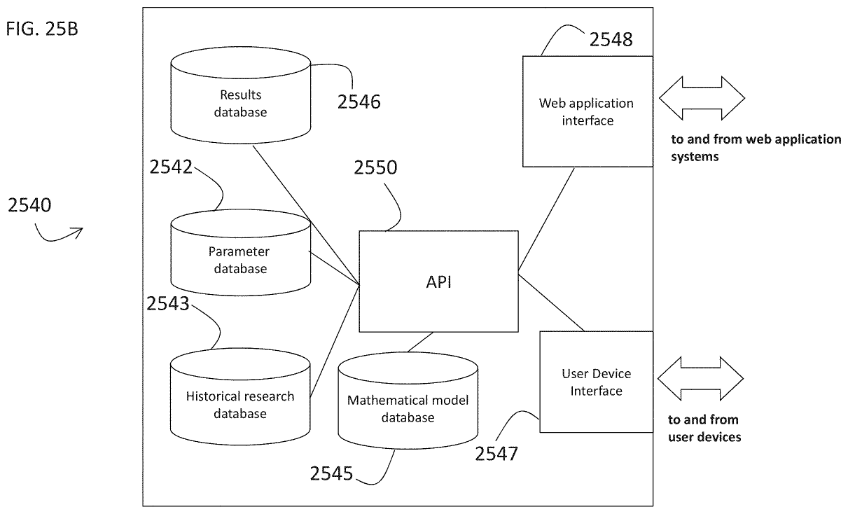

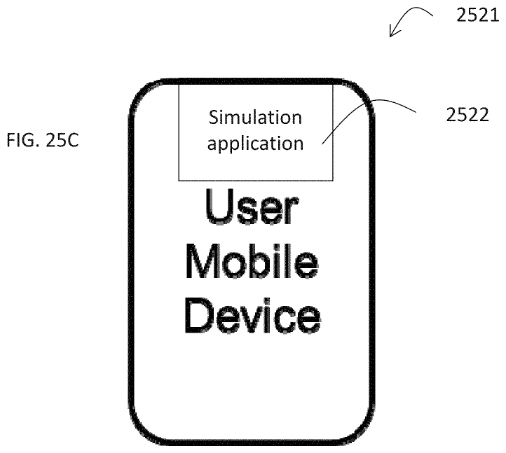

FIG. 16P shows an example embodiment chart of force versus muscle length.

FIG. 16Q shows an example embodiment of a disaccomodated and accommodated eye diagram.

FIG. 16R shows an example embodiment diagram of a simple spring model of ciliary muscle movement.

FIG. 17A shows an example embodiment screenshot of a model of ocular structures for use in simulation.

FIG. 17B shows an example embodiment image of individual ciliary fiber movement during an accommodative process including thickness changes, as indicated by the arrows.

FIG. 17C shows an example embodiment image indicating overall ciliary muscle movement during an accommodative process including changes in thickness, as indicated by the arrows.

FIG. 17D shows an example embodiment diagram of ciliary muscle thickness at ciliary muscle apex versus accommodative amount.

FIG. 17E shows an example embodiment screenshot of a user interface model of ocular structures for use in simulation.

FIG. 17F shows an example embodiment image of ciliary muscle and lens movement during an accommodative process including diameter changes, as indicated by the arrows.

FIG. 17G shows an example embodiment diagram of ciliary muscle ring diameter versus accommodative amount.

FIG. 17H shows an example embodiment diagram of lens diameter versus accommodative amount.

FIG. 17I shows an example embodiment screenshot of a model of ocular structures for use in simulation.

FIG. 17J shows an example embodiment image of forward displacement of lens during an accommodative process, as indicated by arrow.

FIG. 17K shows an example embodiment diagram of forward displacement of the lens versus accommodative amount.

FIG. 17L shows an example embodiment screenshot of a model of ocular structures for use in simulation.

FIG. 17M-17N show example embodiment images of lens thickness changes during an accommodative process, as indicated by the arrows.

FIG. 17O shows an example embodiment diagram of lens thickness changes versus accommodative amount.

FIGS. 17P-17Q show example embodiment screenshots of an accommodated eye and unaccommodated eye model of ocular structures for use in simulation, respectively.

FIGS. 17R-17S show example embodiment diagrams of changes to ciliary muscle and lens respectively, before, midway, and after an accommodative process.

FIG. 17T shows an example embodiment of a user interface diagram displaying measured results of positioning information during a simulation.

FIG. 18A shows an example embodiment of a 3-dimensional cross-sectional model structure diagram showing pre-tensioning of zonules and changes in the lens and ciliary body of an eye.

FIG. 18B shows an example embodiment of a chart showing accommodation of model results as a line using a 3-dimensional cross-sectional model, as compared with a prior art model that captured data points.

FIG. 19A shows an example embodiment of a 3-dimensional cross-sectional model structure diagram 1900 showing simulated accommodation of an eye through ciliary muscle contracting with varied muscle activation.

FIG. 19B shows an example embodiment of 3-dimensional cross-sectional model structure diagram showing simulated accommodation of an eye through longitudinal ciliary fiber contraction and its associated muscle fiber trajectories.

FIG. 19C shows an example embodiment of 3-dimensional cross-sectional model structure diagram showing simulated accommodation of an eye through ciliary contraction with varied muscle activation, particularly showing muscle fiber trajectories for radial fibers.

FIG. 19D shows an example embodiment of 3-dimensional cross-sectional model structure diagram showing simulated accommodation of an eye through ciliary contraction with varied muscle activation, particularly showing muscle fiber trajectories for circular fibers.

FIG. 20A shows an example embodiment of a chart showing accommodation of model results using a 3-dimensional cross-sectional model structure diagram showing as compared with a prior art model for anterior displacement of a lens in millimeters.

FIG. 20B shows an example embodiment of a chart showing accommodation of model results using a 3-dimensional cross-sectional model structure diagram showing as compared with a prior art model for apex thickness of ciliary muscle in millimeters.

FIG. 21 shows an example embodiment of a cross-sectional ocular structure diagram 2160 showing ocular structures of a human eye.

FIG. 22A shows an example embodiment diagram of treatment regions from a particular three zone model protocol.

FIG. 22B shows an example embodiment diagram of treatment regions from a particular three zone model protocol.

FIG. 22C shows an example embodiment diagram of a simulated medical treatment of an eye.

FIG. 22D shows an example embodiment diagram of a simulated medical treatment of an eye, including treatment regions from a particular three zone model protocol.

FIG. 22E shows an example embodiment diagram of a simulated medical treatment of an eye, including treatment regions from a particular three zone model protocol.

FIG. 22F shows an example embodiment chart of macro results of therapy simulation methods.

FIG. 22G shows an example embodiment chart of apex thickness of the ciliary body for various zones simulated, along with a baseline.

FIG. 22H shows an example embodiment chart of length shortening of the ciliary body for various zones simulated, along with a baseline.

FIG. 22I shows an example embodiment chart of micro results for therapy simulation methods.

FIG. 22J shows an example embodiment diagram of different characteristics of pore density that can be changed. First is depth, pore width, and quantity.

FIG. 23 shows an example embodiment diagram of treated stiffness including modulus of elasticity of sclera in a treated region versus volume fraction or percent of sclera volume removed in the treated region for the simulation.

FIG. 24A shows an example embodiment diagram of a simulated medical treatment of an eye, including treatment regions from a particular five zone model protocol.

FIG. 24B shows an example embodiment chart of macro results of therapy simulation methods.

FIG. 24C shows an example embodiment chart of apex thickness of the ciliary body for various zones simulated, along with a baseline, and results that affect scleral stiffness only.

FIG. 24D shows an example embodiment chart of length shortening of the ciliary body for various zones simulated, along with a baseline, and results that affect scleral stiffness only.

FIG. 24E shows an example embodiment chart of macro results of therapy simulation methods and results that affect scleral stiffness and attachment.

FIG. 24F shows an example embodiment chart of apex thickness of the ciliary body for various zones simulated, along with a baseline, and results that affect scleral stiffness and attachment.

FIG. 24G shows an example embodiment chart of length shortening of the ciliary body for various zones simulated, along with a baseline, and results that affect scleral stiffness and attachment.

FIG. 24H shows an example embodiment chart of effects of treatment density on ciliary deformation in accommodation that affect scleral stiffness only.

FIG. 24I shows an example embodiment chart of apex thickness of the ciliary body for various zones simulated versus volume faction percent removed.

FIG. 24J shows an example embodiment chart of length shortening of the ciliary body for various zones simulated versus volume faction percent removed.

FIG. 24K shows an example embodiment chart of effects of treatment density on ciliary deformation in accommodation that affect scleral stiffness and attachment.

FIG. 24L shows an example embodiment chart of apex thickness of the ciliary body for various zones simulated versus volume faction percent removed.

FIG. 24M shows an example embodiment chart of length shortening of the ciliary body for various zones simulated versus volume faction percent removed.

FIG. 25A is an example embodiment of a basic network setup diagram.

FIG. 25B is an example embodiment of a network connected server system diagram.

FIG. 25C is an example embodiment of a user mobile device diagram.

### DETAILED DESCRIPTION

Before the present subject matter is described in detail, it is to be understood that this disclosure is not limited to the particular embodiments described, as such may vary. It should also be understood that the terminology used herein is for the purpose of describing particular embodiments only, and is not intended to be limiting, since the scope of the present disclosure will be limited only by the appended claims.

Accommodation of a human eye occurs through a change or deformation of the ocular lens when the eye transitions from distant focus to near focus. This lens change is caused by contraction of intraocular ciliary muscles that make up the ciliary body, which relieves tension on the lens through suspensory zonule fibers and allows the thickness and surface curvature of the lens to increase. The ciliary muscle can have a ring-shape and can be composed of three uniquely oriented ciliary fiber groups that contract toward the center and anterior of the eye. These three ciliary fiber groups are known as longitudinal, radial and circular. Deformation of the ciliary muscle due to the contraction of the different muscle fibers translates into or otherwise causes a change in tension to the surface of the ocular lens through zonule fibers, whose complex patterns of attachment to the lens and ciliary muscle dictate the resultant changes in the lens during accommodation. Ciliary muscle contraction also applies biomechanical strain at the connection locations between the ciliary muscle and the ocular sclera, known as the white outer coat of the eye. Additionally, biomechanical compression, strain or stress can be caused during accommodation can occur at connection locations between the ciliary muscle and the choroid, known as the inner connective tissue layer between the sclera and ocular retina. Ciliary muscle contraction can also cause biomechanical forces on the trabecular meshwork, lamina cribrosa, retina, optic nerve and virtually every structure in the eye.

Applying the techniques and models described with respect to the various embodiments herein, can lead to outputs and results that fall within known ranges of accommodation of a young adult human, as described in existing medical literature. This verifies the validity of the models with respect to the application of variables due to displacement and deformation of the ocular lens and ciliary muscle.

3D Mathematical Models can incorporate mathematics and non-linear Neohookean properties to recreate behavior of the structures of biomechanical, physiological, optical and clinical importance. Additionally, 3D FEM Models can incorporate data from imaging, literature and software relating to the human eye.

Visualization of accommodation structures is included in addition to means for measuring, evaluating and predicting Central Optical Power (COP). These can be used to simulate and view age specific whole eye structures, optics, functions and biomechanics. Further, they can independently simulate properties of the ciliary muscle, extra-lenticular and lenticular movements of the ocular lens and functions on the ocular lens. Individual simulations of anatomical structures and fibers can reveal biomechanical relationships which would otherwise be unknown and undefined. Numerical simulation of the patient's eye can be created using 3D FEM meshing to accomplish these operations.

To elaborate, representative 3D geometry of resting ocular structures can be computationally defined based on extensive review of literature measurements and medical images of the anatomy of young adult eyes. Then, specialized methods implanted in software, such as AMPS software (AMPS Technologies, Pittsburgh, Pa.), can be used to perform geometric meshing, material property and boundary conditions definitions, and finite element analysis. Ciliary muscle and zonules can be represented as a transverse isotropic material with orientations specified to represent complex fiber directions. Additionally, computational fluid dynamic simulations can be performed in order to produce fiber trajectories, which can then be mapped to the geometric model.

Initially, a lens can begin in a relaxed configuration, before being stretched by pre-tensioning zonule fibers to an unaccommodated position and shape. Unaccommodated lens position can be reached when zonules are shortened to between 75% and 80% of their starting length, and more particularly to about 77% of their starting length, as shown in FIG. 18A. Then accommodative motion can be simulated by performing active contraction of the various fibers of the ciliary muscle. In some embodiments, this can be accomplished using previous models of skeletal muscle that are modified to represent dynamics particular or otherwise specific or unique to the ciliary muscle. Model results representing lens and ciliary anterior movement and deformed ocular lens thickness at a midline and apex can be validated or otherwise verified by comparing them to existing medical literature measurements for accommodation. In order to investigate contributions of the various different ciliary fiber groups to the overall action of the ciliary muscle, simulations can be performed for each fiber group by activating each in isolation while others remain passive or otherwise unchanged.

Various beneficial aspects of the embodiments described herein with respect to the various FIGs. are described with respect to pre-tensioning zonules models and contracting ciliary muscle models.

With respect to the pre-tensioning zonules, modeling can include: 1) Creation of 3D material sheets oriented between measured zonular attachment points of insertion on the lens and origination on the ciliary/choroid; 2) specified fiber direction in the plane of the sheet (i.e. fibers directed from origin to insertion); and 3) Transversely isotropic constitutive material with tension development in the preferred direction. Further, with particular respect to 3), advantages have been achieved, including: a) Time-varying tension parameter input regulates the stress developed in the material; b) Time-varying tension input is tuned to produce required strain in the lens to match measurements of the unaccommodated configuration; c) Age variation in material properties and geometries to produce age-related impact; and d) others.

With respect to the contracting ciliary muscle models, modeling can include: 1) Modified constitutive model to represent smooth and skeletal aspects of ciliary mechanical response, including contraction that affects accommodation and effects on pre-tensioned state of the lens in an unaccommodated configuration; 2) 3 sets of specified fiber directions to represent physiological orientation of muscle cells and lines of action of force production; and 3) Transversely isotropic constitutive material with active force development in the preferred direction. Further, with particular respect to 3), advantages have been achieved, including: a) Activation parameter input regulates the active stress developed in the material; b) Activation input is tuned to produce appropriate accommodative response to match literature measurements; c) Activation of individual muscle fiber groups can be varied in isolation to assess contributions to lens strain/stress; d) Activation of individual muscle fiber groups can be varied in isolation to assess contributions to ocular scleral strain/stress; e) Activation of individual muscle fiber groups can be varied in isolation to asses contributions to choroidal strain/stress; and f) others.

In various embodiments, simulation results can be governed by modification of tensioning and activation inputs to the zonule and ciliary materials, as opposed to performing an applied displacement to external node(s) of a mesh.

In various embodiments, three-dimensional circumferential and other force vectors can be simulated for various ocular structures, thus providing different effects and insights into ocular structures and their movement and relation to one another. Boundary conditions for ocular structures and material property values can be changed and their influence determined as well.

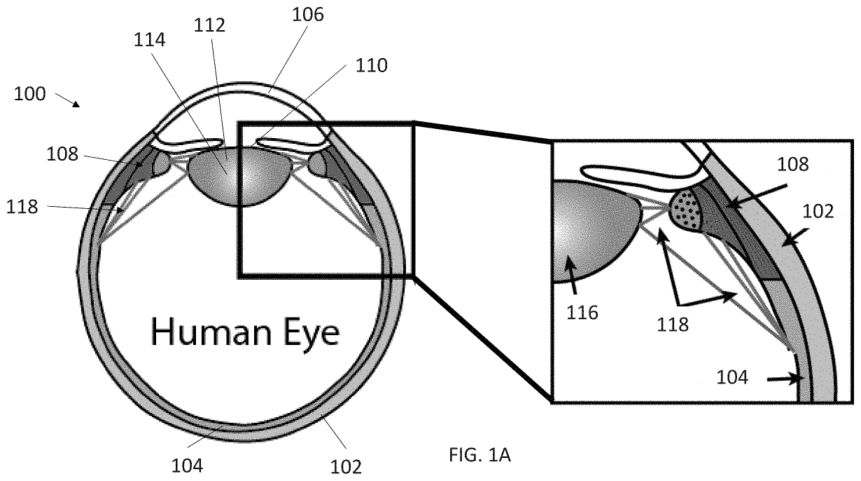

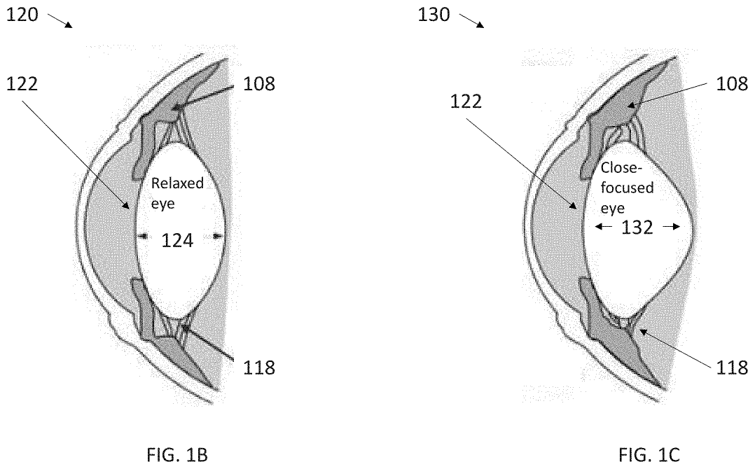

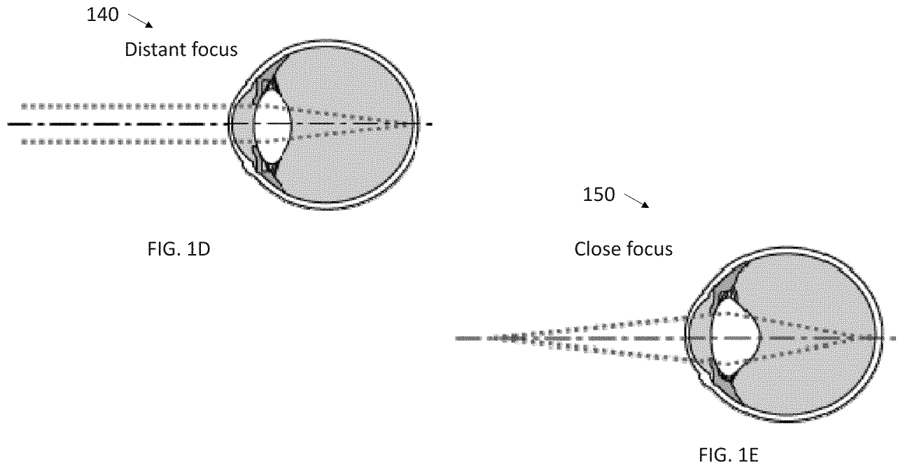

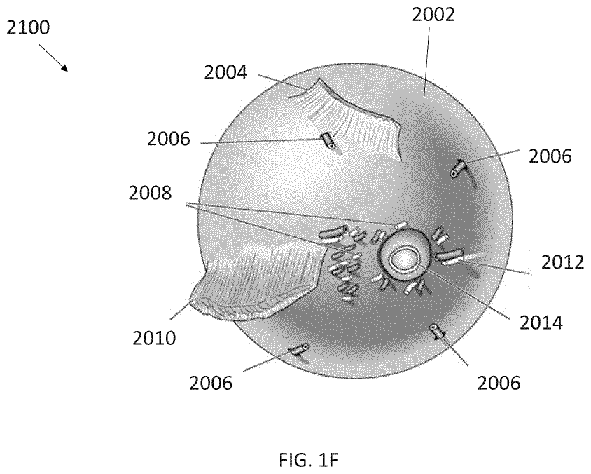

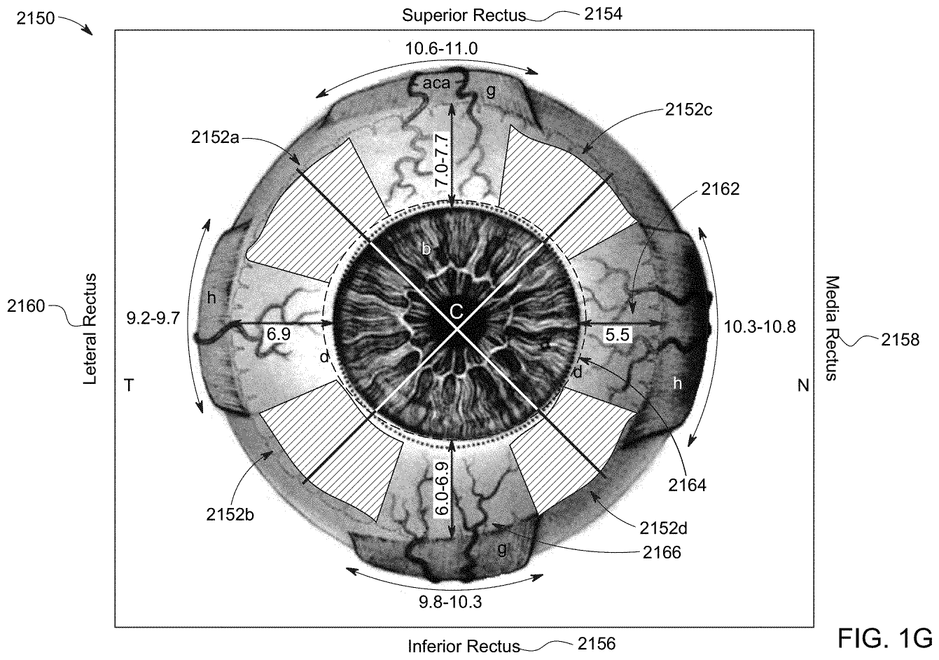

FIG. 1A shows an example embodiment of an anatomical diagram 100 of an eye cross section with a reference key. As shown in the example embodiment, anatomical structures of the eye can include sclera 102; choroid 104; cornea 106; ciliary muscles 108 including circular, radial, and longitudinal fibers; lens 116, including lens capsule 110 and lens nucleus 114; lens cortex 112; and zonules 118 including three anterior, most anterior (MAZ), anterior vitreous, intermediate vitreous, and pars plana.

Accommodation is the process by which the eye changes optical power to focus on objects at various distances by deforming the lens. While age-related changes in the eye have been measured, it was only within the last few years that biomechanics of presbyopia have been brought into focus. Presbyopia causes a loss of accommodative function in the eye, making it harder to focus, especially on near objects or images.

FIGS. 1B-1C show an example embodiment of a cross-section of an eye diagram 120 and 130 illustrating changes in structural components of an eye for distance and near vision respectively. As shown in distance vision diagram 120, for distance vision the eye is relaxed and lens 122 has a lens thickness 124. Ciliary muscles 108 are generally relaxed, and zonules 118 are generally taut. However, as shown in near vision diagram 130, lens 122 changes to lens thickness 132 when the eye attempts to focus on something closer. Lens thickness 132 is greater than lens thickness 124 for a close-focused eye, caused by ciliary muscles 108 contracting and zonules 118 becoming more relaxed.

FIG. 1D shows an example embodiment diagram 140 of how an unaccommodated eye focuses an image through a lens.

FIG. 1E shows an example embodiment diagram of how an accommodated eye focuses an image through a lens.

FIG. 1F shows an example embodiment of an ocular structure diagram 2100 showing ocular structures from a view of the back of a human eye. As shown in the example embodiment, a posterior side of eye 2002 includes a superior oblique insertion 2004, vortex veins 2006, short posterior ciliary arteries and short ciliary nerves 2008, inferior oblique insertion 2010, long posterior ciliary artery and long ciliary nerve 2012, and optic nerve 2014.

FIG. 1G shows an example embodiment of an ocular structure diagram 2150 showing ocular structures from a view of the front or anterior view of a human eye. As shown in the example embodiment, the approximate surface area of an entire ocular globe is about 75 mm. A meridional quadrant 2152a-2152d can be an average surface area of rectus muscles total, about 40.75 mm. As shown, shaded areas or meridional quadrants 2152a-2152d can be target zones for treatment of presbyopia and other conditions using medical techniques and procedures, such as ablation. Oblique quadrants 2152a-2152d can be an average surface area in a target area of about 75 mm-40.75 mm, which equals about 34 mm. As shown, quadrants 2152a-2152d can have different sizes temporal to nasally.

A superior rectus 2154 can be between 10.6 mm and 11 mm, or about 10.8 mm. An inferior rectus 2156 can be between 9.8 mm and 10.3 mm, or about 10.05. A medial rectus 2158 can be between 10.3 mm and 10.8 mm, or about 10.45 mm. A lateral rectus 2160 can be between 9.2 mm and 9.7 mm, or about 9.45 mm. An average combined cornea and limbus 2164 diameter 2162 can be about 12 mm. A distance from limbus 2164 in millimeters can have an approximate range of about 5.5 mm to about 7.7 mm, so for modeling and simulations, a distance of 6 mm can be used. Also shown are anterior ciliary arteries 2166.

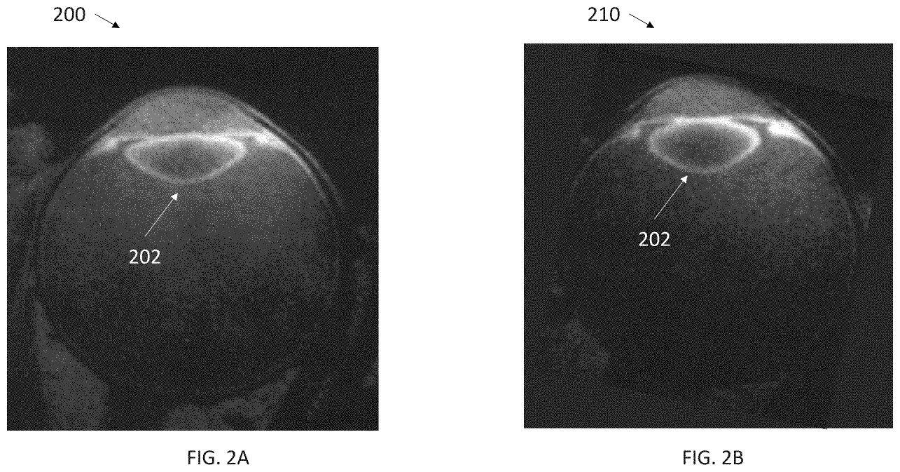

FIGS. 2A-2B shows an example embodiment of an unaccommodated eye cross sectional image 200 and an accommodated eye cross sectional image 210, respectively. As shown in the example embodiments, lens 202 changes from unaccommodated shape with a first thickness to an accommodated shape with a second thickness greater than the first thickness when changing from focusing on distant objects to near objects. The mechanisms underlying this principle are discussed with respect to FIGS. 1B-1C and elsewhere herein.

As discussed previously herein, it would be beneficial to develop improved modeling of ocular structures to better understand ocular mechanisms, including accommodation and disaccommodation. One starting point is to use ocular imaging literature to understand ocular structures and their arrangement with one another.

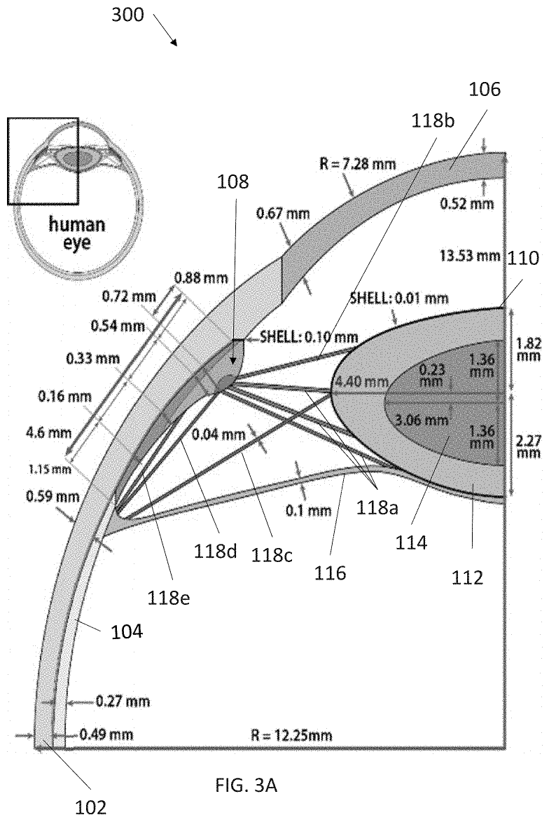

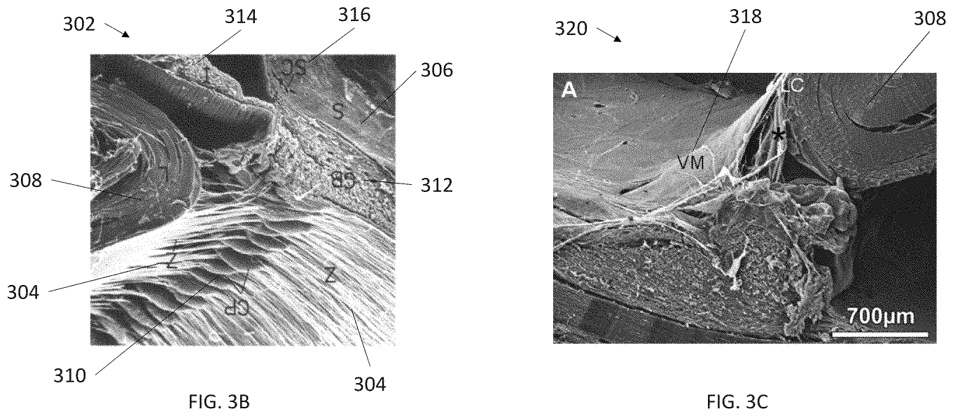

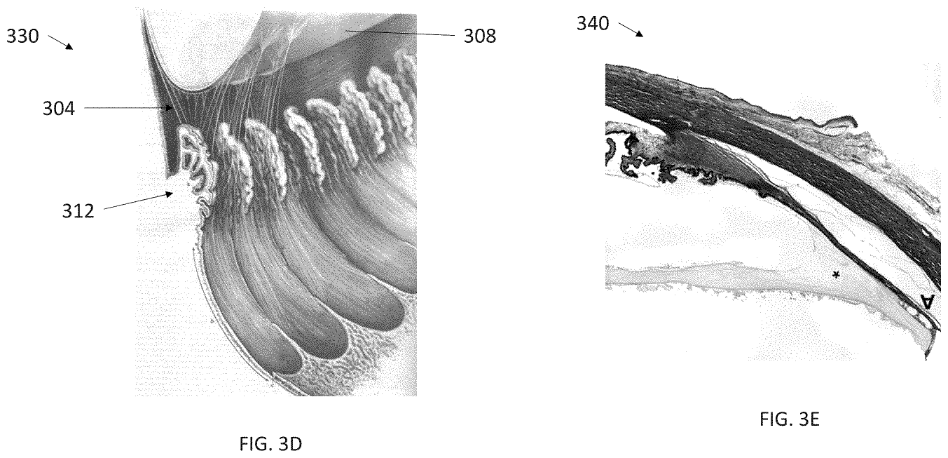

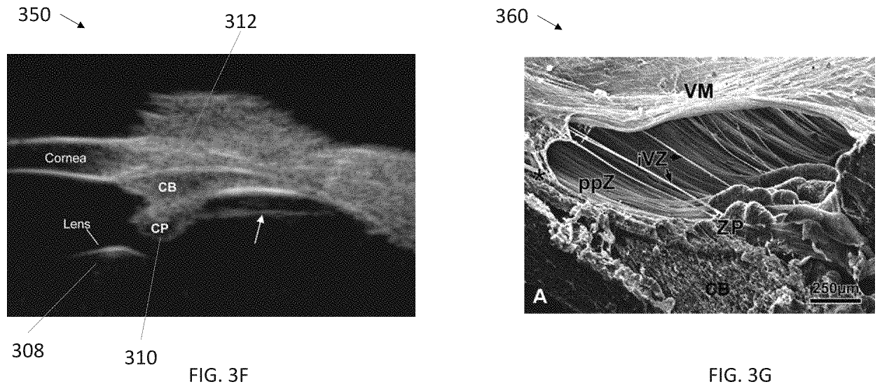

FIG. 3A shows an example embodiment of a cross sectional diagram 300 of an eye based on model structures from existing imaging literature. Like numbers have been included for sclera 102; choroid 104; cornea 106; ciliary muscles 108 including circular, radial, and longitudinal fibers; lens capsule 110; lens nucleus 114; lens cortex 112; and vitreous membrane 116, from FIG. 1A to maintain clarity. Zonules 118 of FIG. 1A are shown individually in FIG. 3A including three anterior zonules 118a, most anterior zonule (MAZ) 118b, anterior vitreous zonule 118c, intermediate vitreous zonule 118d, and pars plana zonule 118e.

Material properties can be defined by various equations and parameter values. Various factors affecting modelling include Neo-Hookean isotropic structures with material and stiffness references, how muscle structure and materials affect models, and how zonule models can be developed with an explanation of transverse isotropy with pre-tensioning.

As shown, various measurements can be implemented in modeling for an eye with a radius of 12.25 mm from a central optical axis to an exterior of sclera 102. Sclera 102 can range in thickness from 0.49 mm to 0.59 mm and choroid 104 can have a thickness of 0.27 mm. Cornea 106 can have a thickness ranging from 0.52 mm to 0.67 mm and a radius of 7.28 mm. A distance from lens capsule 110 to an outer edge of cornea 106 can be about 13.53 mm. Ciliary muscles 108 can have a length of 4.6 mm overall. Lens capsule 110 can be about 0.01 mm thick. Lens cortex 112 and lens nucleus 114 can have a combined radius of about 4.40 mm and a combined thickness of about 4.09 mm. Lens nucleus 114 can have a radius of about 3.06 mm and thickness of about 2.72 mm. Vitreous membrane 116 can be about 0.1 mm thick. Anterior vitreous zonule 118c can be about 0.4 mm thick.

FIG. 3B shows an example embodiment of a Scanning Electron Microscopy image 302 of Zonular fibers 304, and nodal attachments as well as pathway of the zonular proximal and distal insertion zones of an eye, based on model structures from existing imaging literature. Also shown are sclera 306, lens 308, ciliary process 310, ciliary body 312, iris 314, and SC 316.

FIG. 3C shows an example embodiment of a Scanning Electron Microscopy image 320 of Zonular fibers 304 and relationship to the lens 308 and the Vitreous membrane 318 of an eye based on model structures from existing imaging literature.

FIG. 3D shows an example embodiment diagram 330 of a ciliary body 312. In general, ciliary body 312 includes ciliary muscle. Ciliary muscle includes circular fibers, radial fibers, and longitudinal fibers. Ciliary body 312 extends between the iris and the choroid. A cross section of ciliary body 312 has a generally triangular cross section. A base or anterior surface of this triangular cross section is continuous with an iris root. An apex of the triangular cross section is continuous with the choroid and directed posteriorly.

In general, ciliary body 312 includes an anterior surface or base and a posterior surface. The anterior surface is called the pars plicata and can contain about 60-70 different processes. In terms of its location and function within the eye, the anterior surface couples with or attaches lens zonules 304. The posterior surface of ciliary body 312 is called the pars plana. In terms of its location and function within the eye, the posterior surface lies against the sclera . . . . The posterior surface is known to be an important surgical landmark for many medical procedures.

FIG. 3E shows an example embodiment image 340 of a cross-section of the anterior segment of the eye showing the accommodation apparatus and related anatomy as well as the whole eye shell and cornea based on model structures from existing imaging literature.

FIG. 3F shows an example embodiment of an ultrasound biometry image 350 of a cross-section of the anterior segment showing the accommodation apparatus, specifically of the relationship of the ciliary process 310 & ciliary body 312 to the posterior vitreal zonule or pars plana, lens 308, and cornea of an eye, based on model structures from existing imaging literature.

FIG. 3G shows an example embodiment of a Scanning Electron Microscopy image 360 of the relationship between the vitreous membrane, the posterior vitreous zonule insertion and the other zonular structures of an eye based on model structures from existing imaging literature.

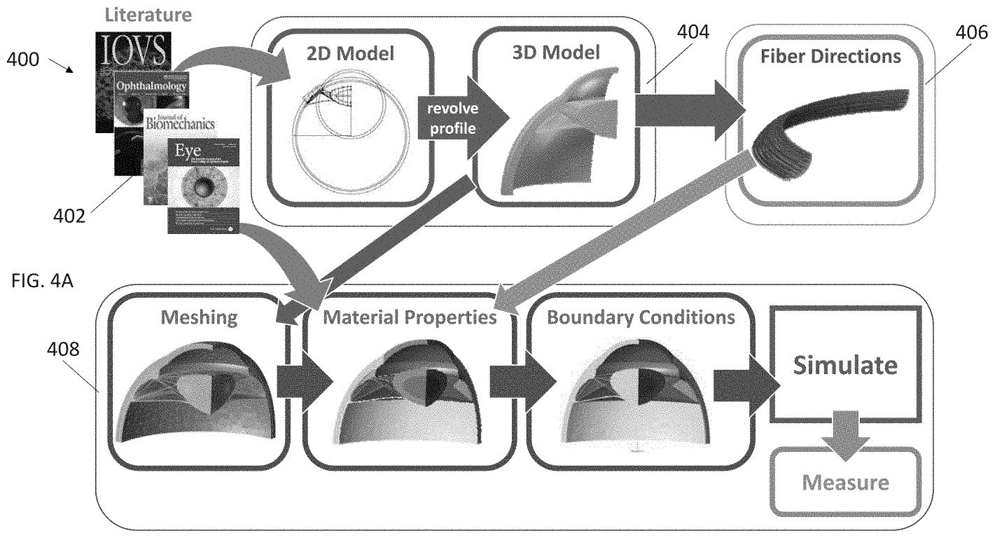

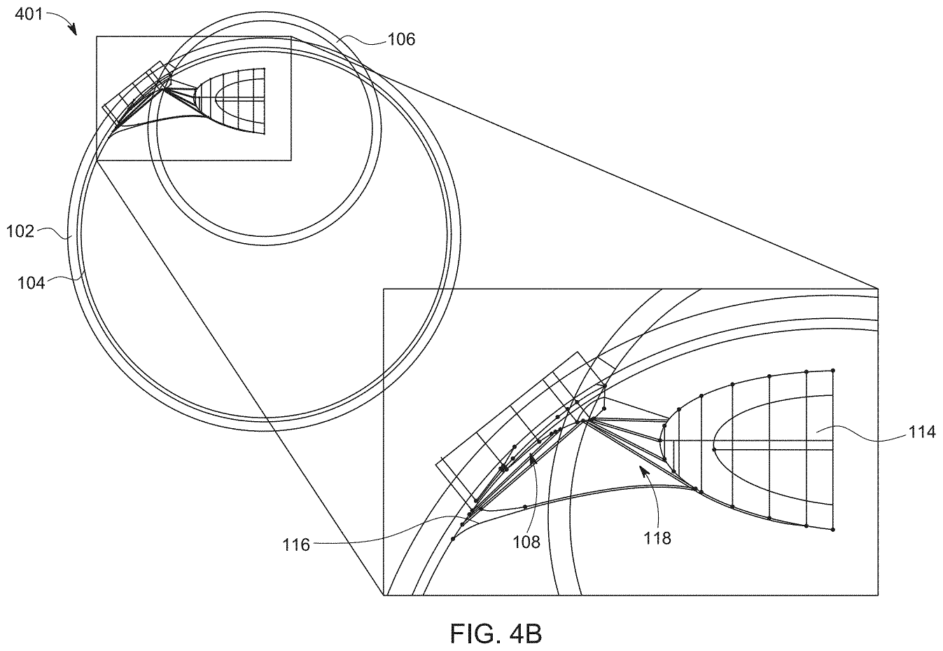

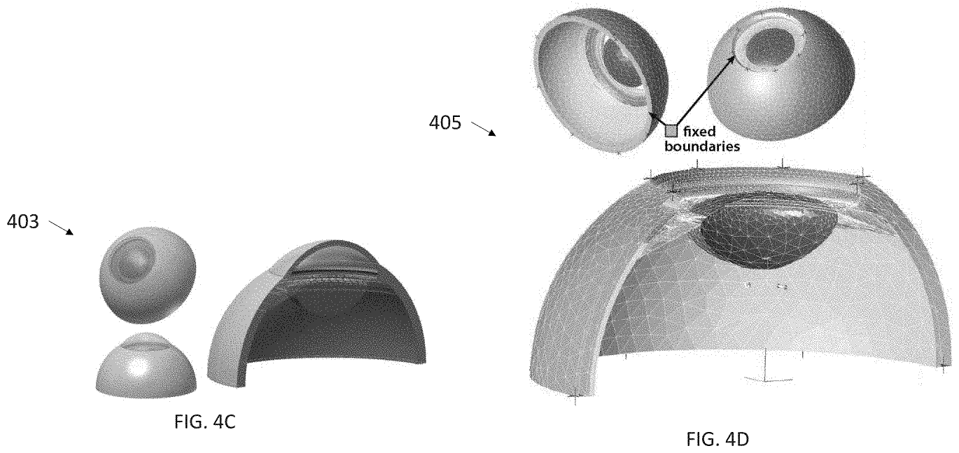

FIG. 4A shows an example embodiment flow diagram 400 of a process of developing new ideas for improved treatments. As shown in the example embodiment, prior research 402, in the form of papers, books, and others, along with mental modeling and known physical laws can be used to develop computational models using different computer programs for generating different models 404. This can also include the use of known physical laws. As shown, these can be two-dimensional models initial, which can then be used to create three-dimensional models. In some embodiments, revolving profiles can lead to improved three-dimensional models. Prior research 402 can also be used to generate structural models 406 of individual ocular structures in various computer programs. As discussed herein, this can include different fiber structural models for fibers of the ciliary body. These computational models 404 and 406 can then be put used in computer simulations 408 along with known physical laws to develop and reveal relationships between structures that may or may not be obvious. Steps such as meshing, inputting and manipulating material properties and boundary conditions can be performed before running the computational simulations and measuring various desired results. As such, simulations can be used to perform “what-if” scenarios in order to generate new ideas, which can be related to or reveal new insights about how to create or improve existing treatments.

To elaborate on the types of computer modeling that can be performed, computer aided design (CAD) programs can generate three-dimensional models of eyes. When inputting the model the computer needs various inputs, including what type of material it is. Examples include stiff, elastic, nonlinear, and others. This may be required for each of the ocular structures. Neo-Hookean types of material models that describes the stress/strain relationships in materials. More simple versions of the model deal with non-linear tissues may also be important. Equations that import material properties for scleras, corneas, and lenses can be used for simulating those tissues' deformation when the ciliary muscle contracts.

These can be unaccommodated or accommodated inputs and allow for modeling to be constructed using measured values and medical images in the existing literature. In the example embodiment, CAD: 3D creation of the Model of unaccommodated 29-year-old eye geometry can be constructed based on literature values and medical images, for example by using Autodesk Inventor computer programs to create geometry and relationships. Once the 3D geometry model is developed, it can then be exported into AMPS which is the finite element analysis (FEA) solver. Other simulations can be used such as Autodesk Simulation CFD and Matlab.

FEA Solvers can be used for automated three-dimensional meshing of solid structures, enter material properties assigned to different components, define boundary conditions, and measure dynamics of accommodation through simulation.

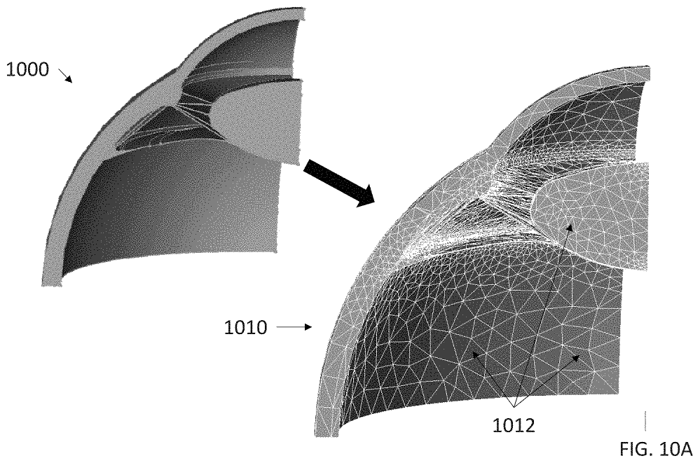

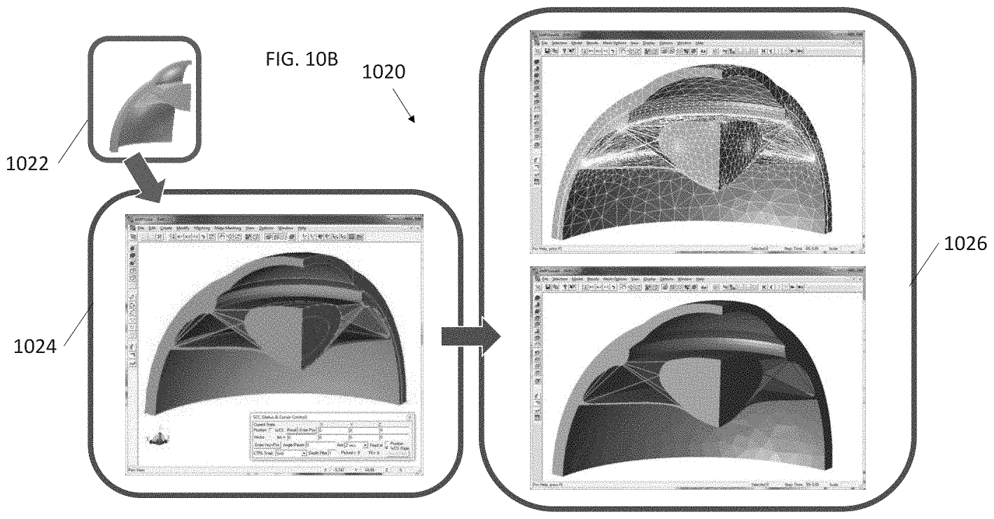

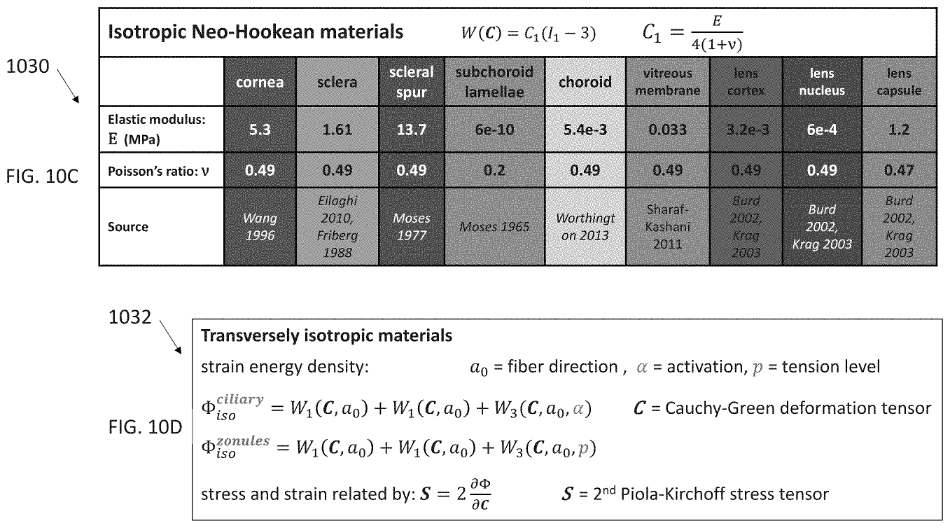

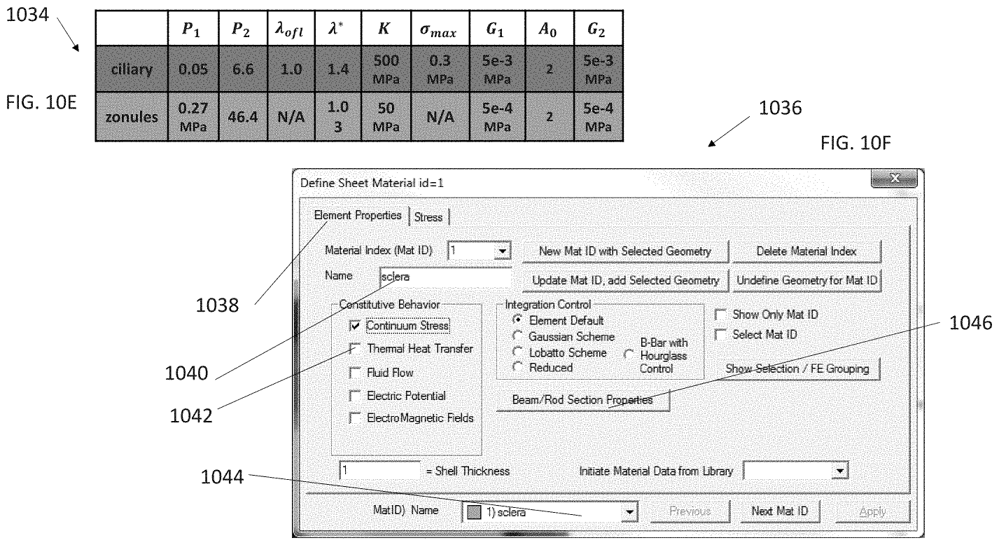

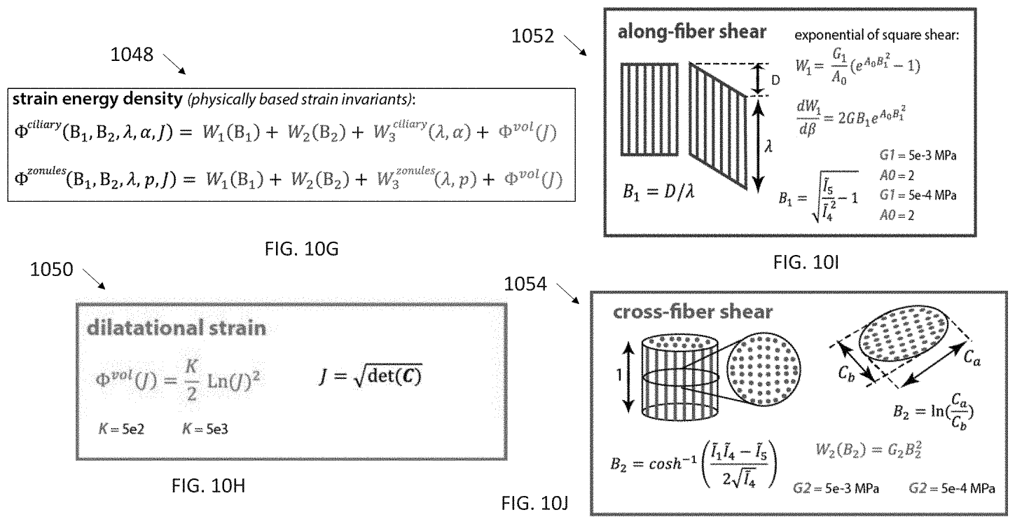

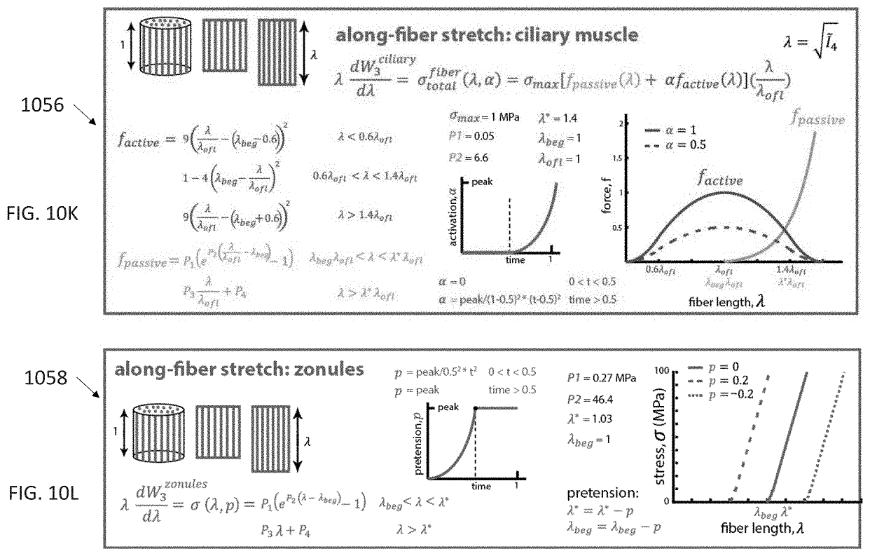

Then there can be an automatic meshing in Amps that fragments complex geometry and is used to solve physics problems, discussed further with respect to FIG. 10. This is an example of simplification of smaller parts or finite element modeling (“FEM”).

A FEM solver is where determination for physics of muscle contraction occurs and then all the corresponding reactions of the other anatomy of the accommodation complex can be determined and analyzed. After the mesh is created material properties can be assigned to each structure and each structure can therefore be understood as a set of elements. Scleral, lens, choroid, zonules, muscle material properties, and others can be unique to the anatomy. Then boundary conditions can be set, and all structures can be fixed at an equator and at the limbus. There is no movement above or below those boundaries after being set. Corneal movement can be legitimately related to the lens. This can be used in a simplified model to understand the lens and the physics of the lens. Although the model may not be perfect, it can still be very useful in determining relationships. Once the mesh and boundary conditions are complete, dynamics simulations can be run.

Finite element analysis can include modeling details: meshing, boundary conditions, and solvers; performing multi-step simulations, such as pre-stretch and muscle contraction for accommodation; and description of measurements.

Another step can occur in which dynamics are determined in order to set up ciliary fiber directions. This is the first attempt to create a 3D modelling of not only the ciliary muscle fiber directions but of actual forces of action of ciliary muscles on the anatomical structures affecting accommodation.

Calibration and validation can also be important. Calibration can be performed using zonule tension modification that may match an average MRI measurement range and ciliary activation that may match an average OCT measurement range of actual subjects. Calibration results for individually tensioned zonules and “tuned” tension can be shown on a bar plot for lens Δradius and Δthickness. Additionally, results for individually activated muscle groups and “tuned” activation can be shown on a bar plot for Δlength and Δthickness

Validation can include a comparison to imaging data of ciliary and lens deformation, which can be simultaneously checked against OCT and MRI averages. Validation results can be shown on a bar plot of A apex thickness and A lens thickness with bars for model and OCT experiments. Similarly, results can be shown on a bar plot of various deformations with bars for model and MRI experiments. These can include A ciliary apex thickness, A lens thickness, A spur to ora serrata distance, forward movement of vitreous zonule insertion zone, forward movement of lens equator, and centripetal lens equator movement.

As a result of validation, contributions of individual zonule sections on lens deformation role of initial lens tension in accommodation. For example, ciliary contraction with no pretension contribution of different muscle fiber groups to ciliary deformation in accommodation influence of ciliary's attachment to the sclera on its function can be examined, along with any differences between “tight” and “loose” attachments.

FIG. 4B shows an example embodiment 401 of a cross sectional diagram for a two-dimensional model design for an eye with enlarged inset to show enhanced detail. Like numbers have been included for sclera 102; choroid 104; cornea 106; ciliary muscles 108 including circular, radial, and longitudinal fibers; lens capsule 110; lens nucleus 114; vitreous membrane 116; and zonules 118 from FIG. 1A and FIG. 3A to maintain clarity.

As shown in the example embodiment, the eye and its various ocular structures can be effectively modeled using a computer modeling program. This can be accomplished by inputting various known structural measurements and structural measurement ranges of lengths, widths, diameters, thicknesses, and others to effectively create a general eye model that can be manipulated in simulations. Additionally, formulas can be developed and implemented based on known relationships between structural components to model different features and interactions. These can then be used to implement the simulations and to model interactions between the various structural components by changing or otherwise manipulating different variables in the formulas to find resulting effects.

FIG. 4C shows an example embodiment diagram 403 of a three-dimensional model of an eye from a perspective view, side view, and side cross-sectional view.

FIG. 4D shows an example embodiment diagram 405 of a three-dimensional meshing model of an eye from a bottom perspective view, top perspective view, and side cross-sectional view. This will be discussed further with respect to FIG. 10.

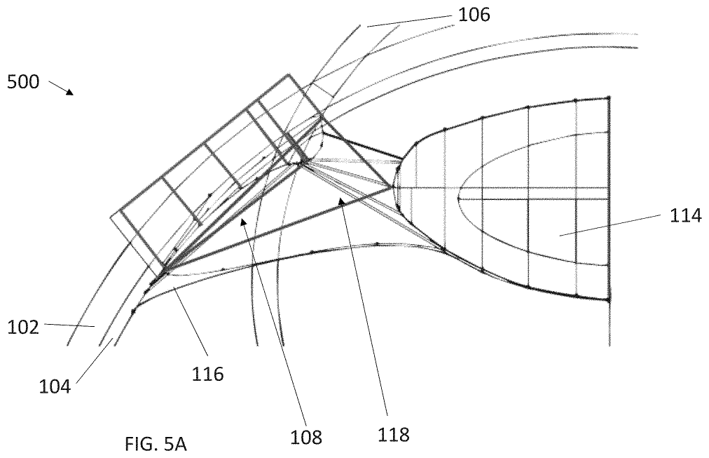

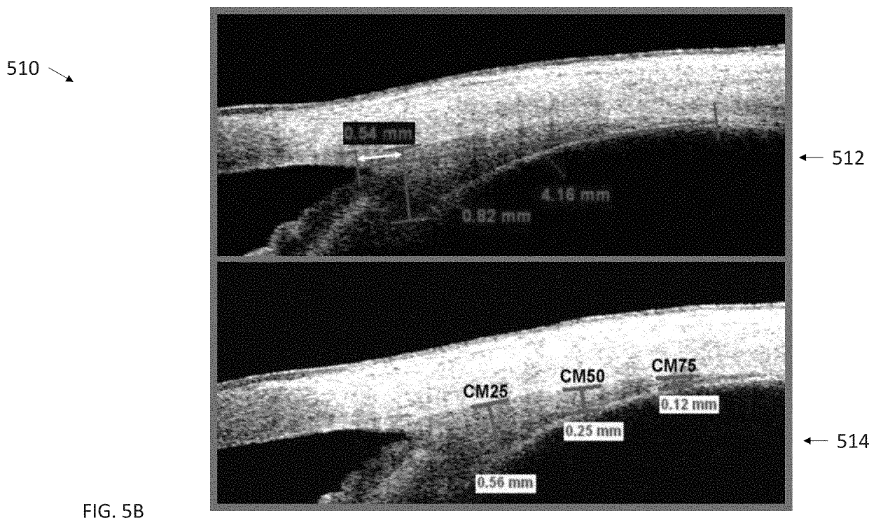

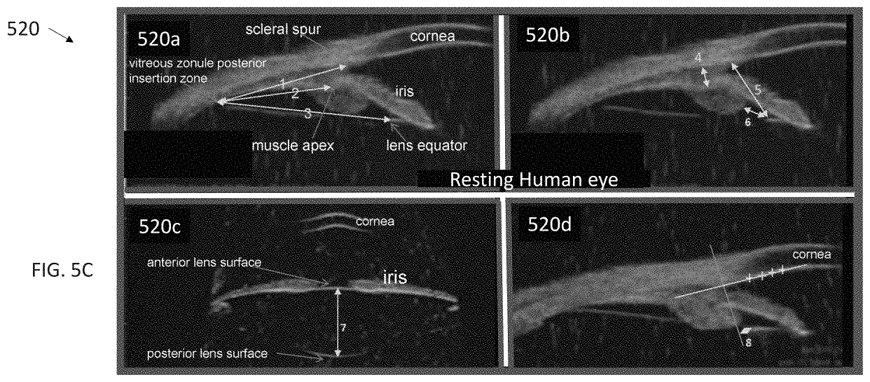

FIG. 5A shows an example embodiment of a two-dimensional cross-sectional diagram 500 for a two-dimensional model design for an eye showing measurements of unaccommodated ocular structures. Like numbers have been included for sclera 102; choroid 104; cornea 106; ciliary muscles 108 including circular, radial, and longitudinal fibers; lens capsule 110; lens nucleus 114; vitreous membrane 116; and zonules 118

FIG. 5B shows an example embodiment of a prior art cross sectional image 510 for a two-dimensional model design for an eye showing measurements of unaccommodated ocular structures. An upper section 512 and lower section 514 show different measurement values of the same unaccommodated eye. As shown in upper section 512, measurements of an unaccommodated eye's ocular structures have shown that a . . . has a length of 0.54 mm and a . . . of 0.82 mm while a . . . has a length of 4.16 mm. As shown in the lower section 514, a ciliary muscle measurement can show a thickness of 0.56 mm at a first point, a thickness of 0.25 mm at an intermediate point, and a thickness of 0.12 mm at a third point. All of these measurements can then be used as two dimensional measurements for a two-dimensional accommodation model. This can be used in developing effective formulas and implemented in simulations.

FIG. 5C shows an example embodiment diagram 520 of prior art cross sectional images 520a-520d for a two-dimensional resting human eye showing measurements of unaccommodated ocular structures. As shown in the example embodiment, measurements of various ocular structures can be conducted for the unaccommodated eye in order to develop an effective 2-dimensional model. Diagram 520a shows measurements from vitreous zonule posterior insertion zone to a scleral spur, muscle apex and lens equator.

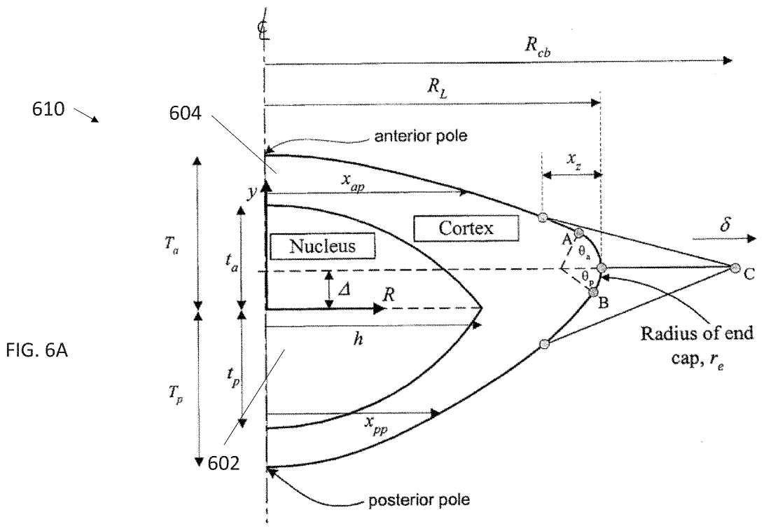

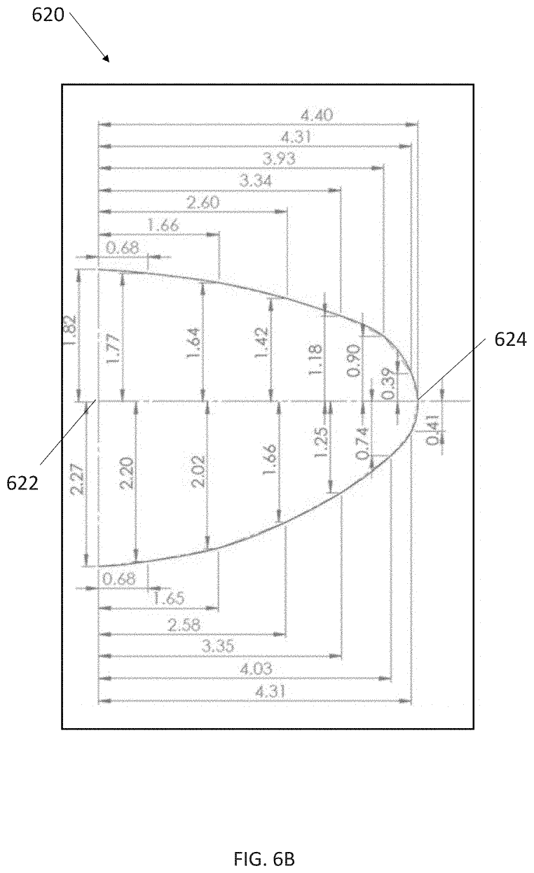

FIG. 6A shows an example embodiment of a cross sectional diagram 610 for a two-dimensional model design for an eye showing variables of accommodated ocular structures. Here, the nucleus 602 and cortex 604 of the lens are modeled and are centered at the origin of the x-y plane. As shown in the example embodiment, various changes can be measured and modeled effectively in a two-dimensional x-y plane to account for all the changes that can occur during accommodation. These can include changes along the x-axis, including: R_{cb}, R_{L}, R, xₐₚ, xₐₚ, x_{z}, h, xₚₚ, and δ. These can also include changes along the y-axis, including: Tₐ, Tₚ, tₐ, tₚ, and Δ. Changes affecting an end cap, rₑ can be measured according to θₐ and θₚ. These and other variables can be used to generate models of the lens and other ocular structures and their relationships.

FIG. 6B shows an example embodiment of a cross sectional diagram 620 for a two-dimensional model design for an eye showing dimensions of accommodated ocular structures. In the diagram, the outward facing surface of the lens is shown above the x-axis while the inward facing surface is below the x-axis. As shown in the example embodiment, standard measurements for x-axis distance and y-axis height of the lens centered at and moving away from the origin 622 toward the end cap 624 for the outward facing surface of the lens have been measured at (0, 1.82), (0.68, 1.77), (1.66, 1.64), (2.60, 1.42), (2.60, 1.18), (3.93, 0.90), (4.31, 0.39), and (4.40, 0). Similarly, standard measurements for x-axis distance and y-axis height of the lens centered at and moving away from the origin 622 toward the end cap 624 for the inward facing surface of the lens have been measured at (0, −2.27), (0.68, −2.20), (1.65, −2.02), (2.58, −1.66), (3.35, −1.25), (4.03, −0.74), and (4.40, 0).

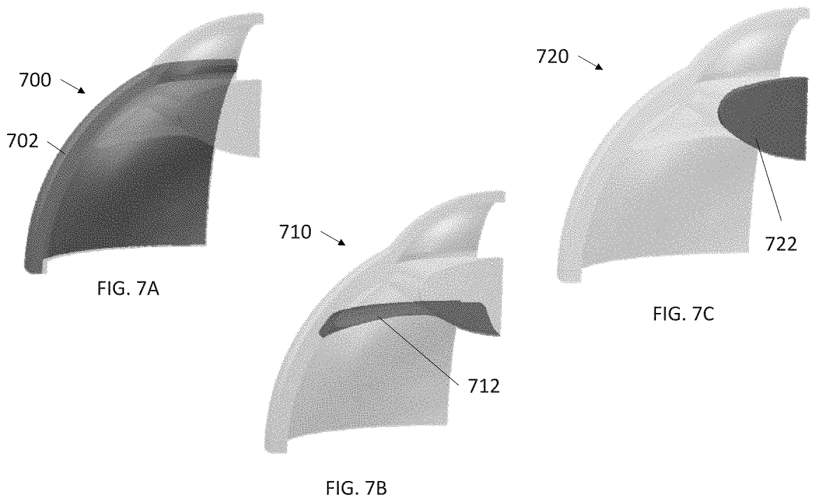

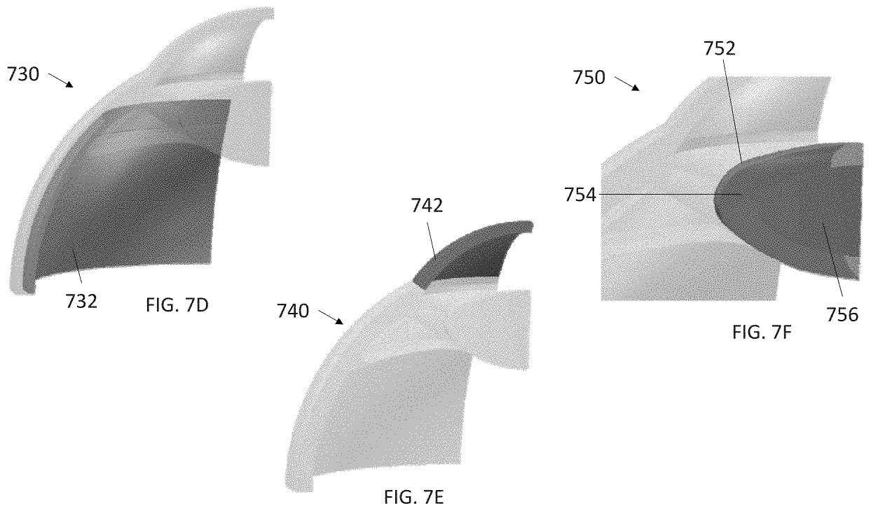

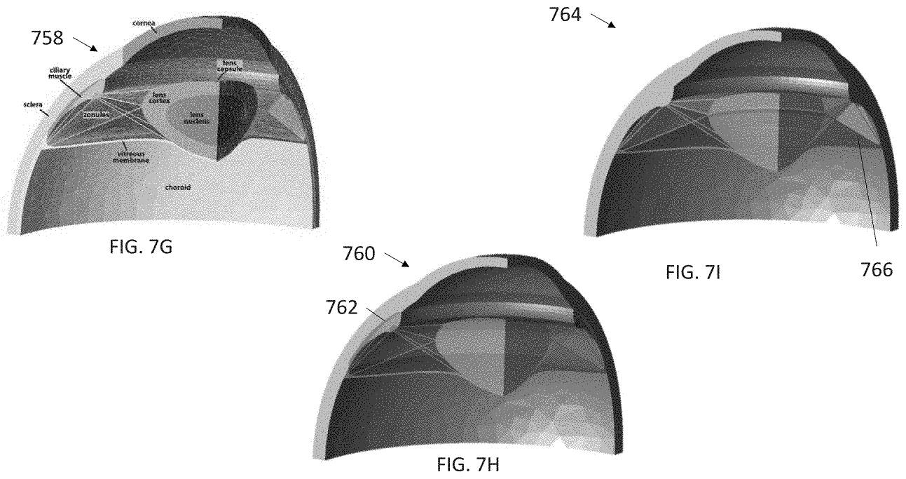

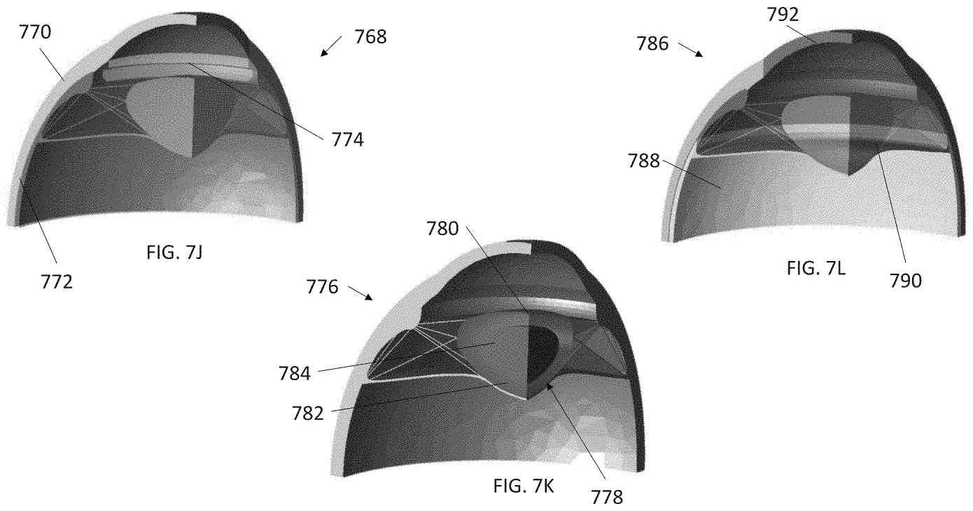

FIG. 7A shows an example embodiment of a cross-sectional 3-dimensional model structure diagram 700 showing a shaded sclera 702 of an eye.

FIG. 7B shows an example embodiment of a cross-sectional 3-dimensional model structure diagram 710 showing a shaded vitreous membrane 712 of an eye.

FIG. 7C shows an example embodiment of a cross-sectional 3-dimensional model structure diagram 720 showing a shaded lens 722 of an eye.

FIG. 7D shows an example embodiment of a cross-sectional 3-dimensional model structure diagram 730 showing a choroid 732 of an eye.

FIG. 7E shows an example embodiment of a cross-sectional 3-dimensional model structure diagram 740 showing a cornea 742 of an eye.

FIG. 7F shows an example embodiment of a cross-sectional 3-dimensional model structure diagram 750 showing a capsule 752, cortex 754, and nucleus 756 of an ocular lens.

FIG. 7G shows an example embodiment of a cross-sectional 3-dimensional model structure diagram 758 showing various ocular structures of an eye.

FIG. 7H shows an example embodiment of a cross-sectional 3-dimensional model structure diagram 760 showing a shaded ciliary muscle 762 of an eye.

FIG. 7I shows an example embodiment of a cross-sectional 3-dimensional model structure diagram 764 showing shaded zonules 766 of an eye.

FIG. 7J shows an example embodiment of a cross-sectional 3-dimensional model structure diagram 768 showing a sclera 770 of an eye. Also shown are subchoroid Lamellae 772 and scleral spur or shell 774.

FIG. 7K shows an example embodiment of a cross-sectional 3-dimensional model structure diagram 776 showing a shaded lens 778 of an eye, including capsule 780, cortex 782, and nucleus 784.

FIG. 7L shows an example embodiment of a cross-sectional 3-dimensional model structure diagram 786 showing a shaded choroid 788, vitreous membrane 790, and cornea 792.

It should be understood that various modeling programs can be used to develop ocular structural models. One example is Autodesk Inventor and another is Autodesk Simulation CFD, both by Autodesk, Inc.

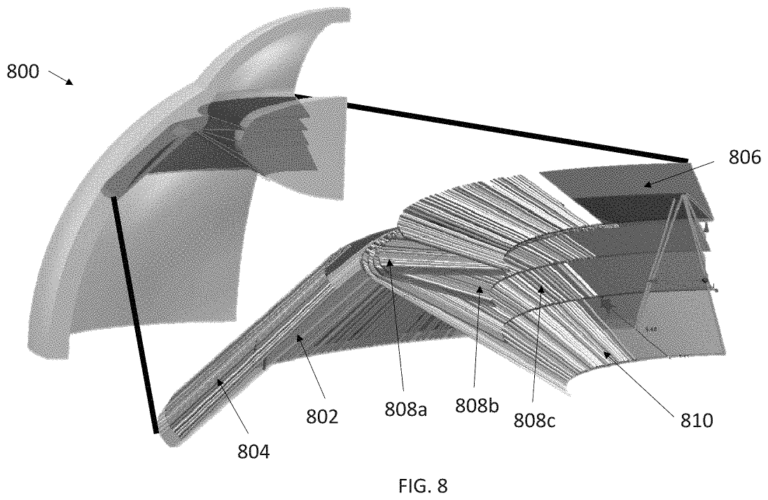

FIG. 8 shows an example embodiment of a cross-sectional 3-dimensional model structure diagram 800 showing a zonules model of an eye with enlarged inset to show enhanced detail. These are shown as various layers including intermediate vitreous zonule layer 802; pars plana zonule layer 804; most anterior zonule (MAZ) layer 806; three anterior zonule layers 808a, 808b, 808c; and anterior vitreous zonule layer 810. As shown, distances can be modeled from a central point in order to ensure modeling accuracy.

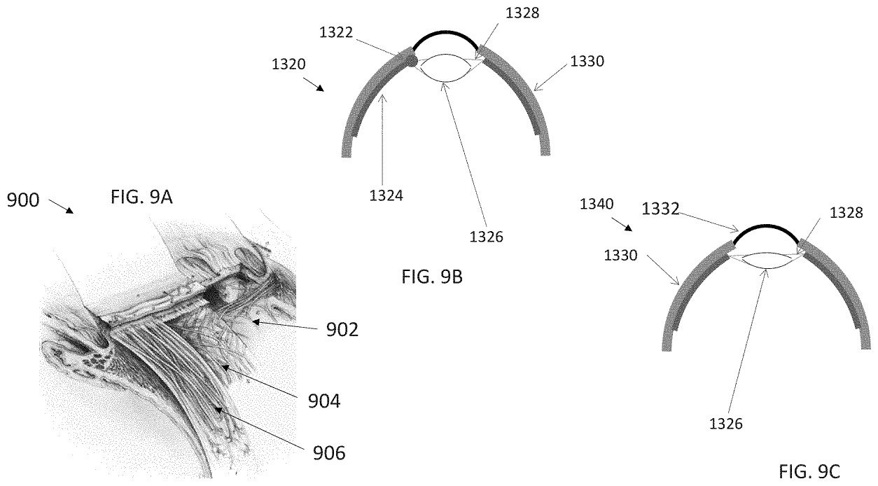

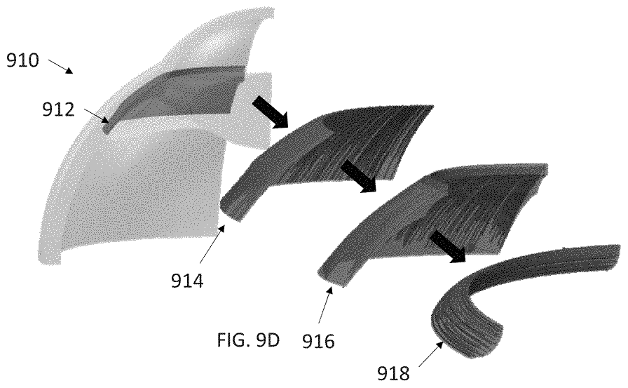

FIG. 9A shows an example embodiment of a prior art diagram 900 of ciliary fibers of an eye. The anatomical structure of ciliary muscles is known to include circular ciliary fibers 902, radial ciliary fibers 904, and longitudinal ciliary fibers 906. These are generally arranged with circular ciliary fibers 902 being the innermost ciliary fibers and arranged in circumferential fashion around a central location. Radial ciliary fibers 904 generally make up an intermediate layer. An outer layer of ciliary fibers are longitudinal ciliary fibers 906, which generally run outward in a radial fashion from a central location.

FIG. 9B shows an example embodiment of an accommodated eye diagram 1320. As the schematic diagram of the eye is shown, major structures involved in accommodation include: a corneo-scleral shell, a crystalline lens, a ciliary body containing ciliary muscles, and the zonular fibers connecting the ciliary body to the crystalline lens. For the accommodated eye a pars plicata portion of a ciliary body 1322 moves upward and inward while ciliary muscle 1324 contracts. Lens 1326 becomes steeper or thicker and leads to higher power for short distance vision. Zonules 1328 are relaxed and sclera 1330 is located exterior to ciliary muscle 1324.

FIG. 9C shows an example embodiment of a disaccomodated eye 1340. Here, cornea 1332 is coupled with sclera 1330. Zonules 1328 become taut and cause lens 1326 to become flatter or thinner, leading to lower power used for long distance vision. As is known in the art, other names for zonules 1328 include: suspensory ligaments, zonules of Zinn, zonular apparatus, and others. Zonular fibers can couple with lens 1326 are known as: anterior, central, and posterior. Ciliary muscle 1324 is contained within a ciliary body.

As shown in FIGS. 9B-9C, a schematic of the eye with the major structures involved in accommodation: the corneo-scleral shell, the crystalline lens, the ciliary body (containing the ciliary muscle), and the zonular fibers connecting the ciliary body to the crystalline lens. The relaxed, or disaccommodated eye is shown on the right. The ciliary muscle is relaxed and the zonules are pulled taut, flattening the lens for distance vision. The accommodated eye is shown on the left. Here, the ciliary muscle is contracted, relaxing the tension on the zonules and allowing the crystalline lens to take its more natural, curved shape for near vision.

FIG. 9D shows an example embodiment of a cross-sectional 3-dimensional model structure diagram 910 showing an integrated composite ciliary fiber model 912 of an eye including an exploded view with separate longitudinal layer model 914, radial layer model 916, and circular layer model 918.

FIG. 10A shows an example embodiment of a cross-sectional 3-dimensional model structure diagram 1000 of an eye with enlarged inset to show a meshing model 1010. Meshing is a technique that is known in modeling to be an effective way of representing three-dimensional structures with computer software. Meshing can include numerous cells 1012 of different sizes and shapes. As shown in the example embodiment, the cells in meshing model 1010 are triangular, although other regular and irregular polygonal shapes can be used. In general, smaller cells allow for closer approximation to any curves of the structure being modeled. As such, here highly rounded areas, such as the side of an ocular lens have smaller cells than comparatively larger round structures, such as a choroid wall. Meshing model 1010 in the example embodiment has been created using AMPS technologies software although many others are known.

FIG. 10B shows an example embodiment diagram 1020 of a meshing process. Here, meshing model geometry for finite element analysis can include using 260927 tetrahedral elements with 1787 triangular shell elements and 111970 nodes. As shown in the example embodiment, once the model has been created in step 1022, for example using Autodesk Inventor, the model can be converted to an intermediate stage 1024, for example in AMPSolid. Then the model can be converted to final meshed model 1026, for example in AMPView64.

FIG. 10C shows an example embodiment chart 1030 of material parameters of ocular structures. As shown in the example embodiment, isotropic Neo-Hookean materials properties of various ocular structures can be based on their elastic modulus E (MPa) and Poisson's ratio. These can be different for the cornea, sclera, scleral spur, subchoroid lamellae, choroid, vitreous membrane, lens cortex, lens nucleus, lens capsule, and other structures.

FIG. 10D shows an example embodiment chart 1032 of various formulas governing transversely isotropic materials.

FIG. 10E shows an example embodiment chart 1034 of parameters for ciliary muscle and zonules.

Various formulas and definitions used in modeling and simulation include: array size=side length of the square area of treatment (mm); treated surface area=surface area of sclera where treatment is applied (mm\hat{ }2); treated surface area=array²; thickness=thickness of sclera in the treated area (mm), assumed uniform; treated volume=volume of sclera where treatment is applied (mm\hat{ }2); treated volume=treated surface area*thickness=array²*thickness; density %=percent of treated surface area occupied by pores (%); spot size=surface area of one pore (mm\hat{ }2); # pores=number of pores in the treated region;

$$\# pores = \frac{density \% * treated surface area}{spot size * 100} = \frac{density \% * {array}^{2}}{spot size * 100} * round to nearest whole number ; total pore surface area = total area within the treated surface area occupied by pores ; total pore surface area = spot size * \# pores \approx \frac{density \% * treated surface area}{100} \approx \frac{density \% * {array}^{2}}{100} ;$$

depth=depth of one pore (mm); dependent on pulse per pore (ppp) parameter; depth %=percent of the thickness extended into by the pore depth (%);

$$depth \% = \frac{depth}{thickness} * 100 ; total pore volume = total area within the treated surface area occupied by pores ; total pore volume = total pore surface area * depth = spot size * \# pores * depth \approx \frac{density \% * treated surface area * depth}{100} \approx \frac{density \% * {array}^{2} * depth}{100} ;$$

volume fraction=percent of treated volume occupied by pores (%), i.e. percent of sclera volume removed by the laser; and

$$volume fraction = \frac{total pore volume}{treated volume} * 100 \approx \frac{density \% * depth}{thickness} = \frac{density \% * depth \%}{100} .$$

Array size, density %, spot size, depth, pulse per pore, and others can be parameters of a laser treatment. Thickness and others can be properties of the sclera. Inputs to calculate new stiffness can include volume fraction and others.

Further, calculating the new stiffness of a sclera in a treated region can be based on various factors including: volume fraction=percent of treated volume occupied by pores (%), i.e. percent of sclera volume removed by the laser;

$$volume fraction = \frac{total pore volume}{treated volume} * 100 \approx \frac{density \% * depth}{thickness} = \frac{density \% * depth \%}{100} ;$$

stiffness=modulus of elasticity of sclera before treatment (MPa); treated stiffness=modulus of elasticity of sclera after treatment (MPa); estimated from microscale mixture model; and

$$treated stiffness = ( 1 - \frac{volume fraction}{100} ) * stiffness \approx ( 1 - \frac{density \% * depth}{thickness * 100} ) * stiffness = ( 1 - \frac{density \% * depth \%}{10000} ) * stiffness .$$

Input parameters to a finite element model of treated zones can be treated stiffness.

Information from FIGS. 10C-10E can be modified and measured in various embodiments to determine effects and changes. This can be done in AMPS software including AMPView64 and others.

FIG. 10F shows an example embodiment of a user interface screen 1036 for modifying various parameters during modeling. Here, users can navigate using tabs 1038, enter information using fields 1040, select buttons 1042 that control different aspects of the model, select different drop-down menus 1044, and execute computer controlled processes stored in memory by selecting buttons 1046.

FIG. 10G shows an example embodiment chart 1048 of strain energy density equations for ciliary muscle and zonules. These can be physically based strain invariants.

FIG. 10H shows an example embodiment chart 1050 of dilational strain equations.

FIG. 10I shows an example embodiment chart 1052 of along-fiber shear equations and diagram.

FIG. 10J shows an example embodiment chart 1054 of cross-fiber shear equations and diagrams.

FIG. 10K shows an example embodiment chart 1056 of along-fiber stretch equations and diagrams for ciliary muscles, including activation versus time and force versus fiber length.

FIG. 10L shows an example embodiment chart 1058 of along-fiber stretch equations and diagrams for zonules, including pretension versus time and stress versus fiber length.

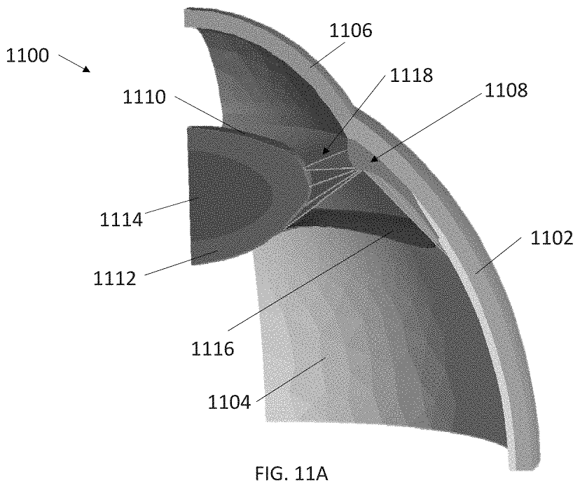

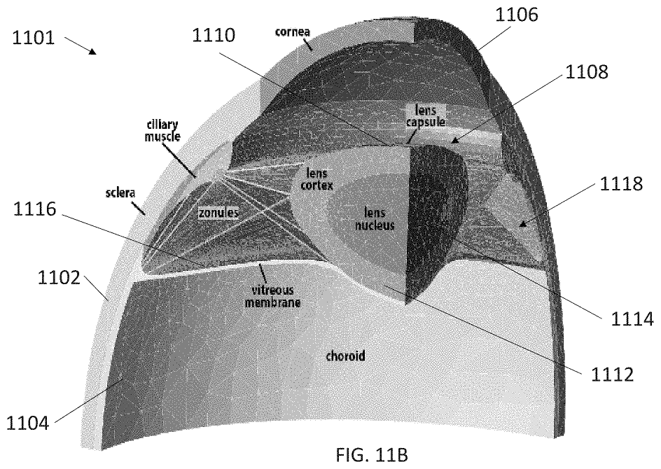

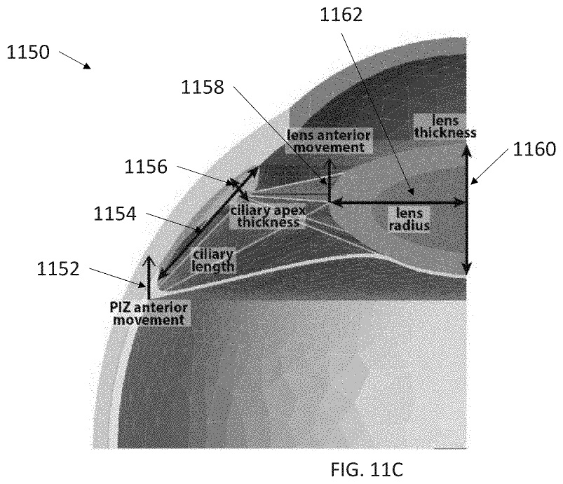

FIG. 11A shows an example embodiment perspective view of a cross-sectional three-dimensional model structure diagram 1100 of an eye. When creating a three-dimensional model an initial step can be to define different structures. Here, ocular structures are being modeled. As such, each ocular structure is first defined as sclera 1102; choroid 1104; cornea 1106; ciliary muscles 1108; lens capsule 1110; lens cortex 1112; lens nucleus 1114; and vitreous membrane 1116, and zonules 1118.

FIG. 11B shows an example embodiment perspective view of a cross-sectional three-dimensional model structure diagram 1101 of an eye. Each ocular structure is first defined as sclera 1102; choroid 1104; cornea 1106; ciliary muscles 1108; lens capsule 1110; lens cortex 1112; lens nucleus 1114; and vitreous membrane 1116, and zonules 1118.

FIG. 11C shows an example embodiment side view of a cross-sectional three-dimensional model structure diagram 1150 of an eye. Each ocular structure is first defined as sclera 1102; choroid 1104; cornea 1106; ciliary muscles 1108; lens capsule 1110; lens cortex 1112; lens nucleus 1114; and vitreous membrane 1116, and zonules 1118. Here, movement, dimensions and thicknesses are shown. Modelling, requires various dimensions, defining descriptions of included ocular structures in the form of geometry reference, and explaining required simplifications.

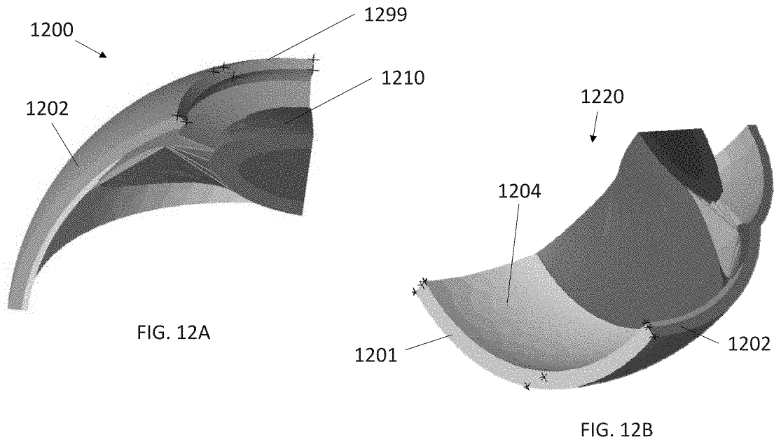

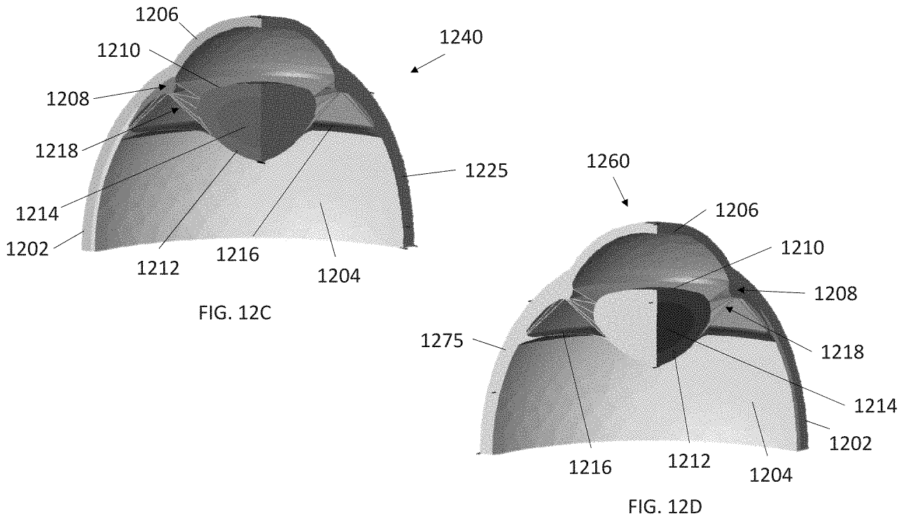

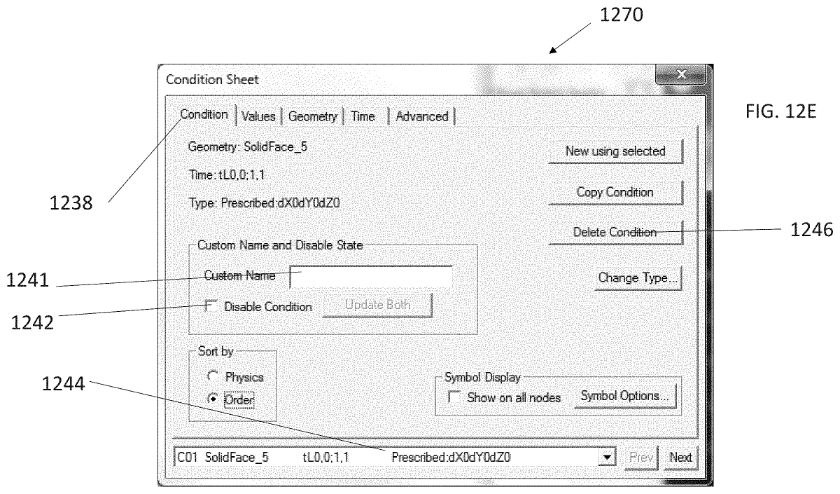

FIGS. 12A-12B show an example embodiment of a cross-sectional three-dimensional model structure diagram 1200, 1220 with upper and lower boundaries of an eye, respectively. After defining different structures in three-dimensional modeling, it can be important to define boundary positions. When modeling for changes in accommodation the entire ocular structure does not need to be modeled since portions of the back of the eye do not play a part in accommodative function. As such, areas near the lens are most important. Thus, defining boundary positions that are near the lens is useful in constraining any modeling and later simulations that may utilize the model.

As shown, the exterior structures are those which require boundary placement since they are the ones at the far extremes of the model. Here, the exterior structures that are constrained by selection of boundaries include sclera 1202 and choroid 1204. An upper boundary 1299 around a semi-circular area of sclera 1202 above the outward facing lens capsule 1210 is set to conserve modeling resources as shown in FIG. 12A. A rotation of the model in FIG. 12B shows a lower boundary 1201 affecting sclera 1202 and choroid 1204 in the rotated cutaway view. Boundary conditions can be fixed in the x, y, and z-directions in FIGS. 12A-12B.

FIGS. 12C-12D shows an example embodiment of a cross-sectional three-dimensional quarter model structure diagram 1240, 1260 of an eye with radial symmetry and having a right and left boundary, respectively. As shown in the models, no out of plane translation is allowed to occur due to left and right boundary setting. FIG. 12C shows how boundaries can be fixed in the x-direction, while FIG. 12D shows how boundaries can be fixed in the z-direction.

As shown, each of the modeled ocular structures requires boundary placement on the left and right here since each is being limited at the edge of the model. Here, the structures that are constrained by selection of boundaries include sclera 1202; choroid 1204; cornea 1206; ciliary muscles 1208; lens capsule 1210; lens cortex 1212; lens nucleus 1214; and vitreous membrane 1216, and zonules 1218. A right planar boundary 1225 along the desired plane is set to conserve modeling resources as shown in FIG. 12C. A left planar boundary 1275 along the desired plane is set to conserve modeling resources as shown in FIG. 12D. Global pressure on interior surfaces (2e-3 MPa) can be set at Intraocular pressure (IOP)—15 mmHg.

FIG. 12E shows an example embodiment of a user interface screen 1236 for modifying various parameters during modeling. Here, users can navigate using tabs 1238, enter information using fields 1241, select buttons 1242 that control different aspects of the model, select different drop-down menus 1244, and execute computer controlled processes stored in memory by selecting buttons 1246.

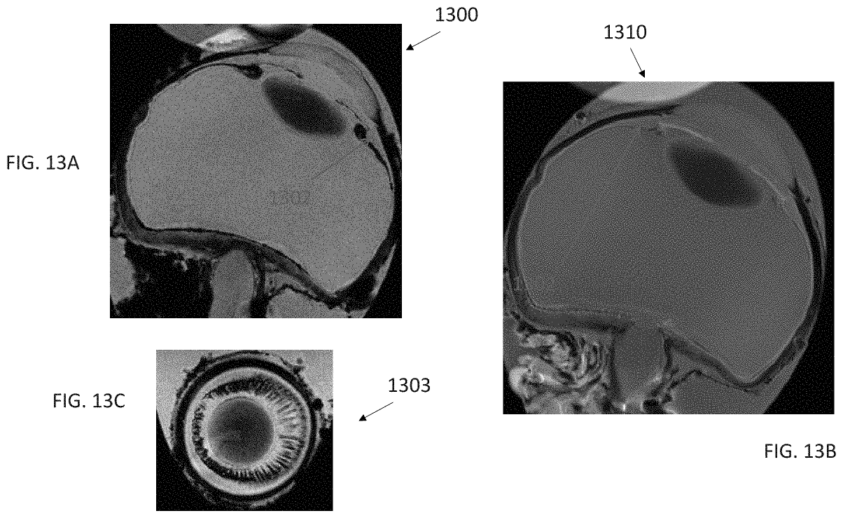

FIG. 13A shows an example embodiment of a cross-sectional 7T MRI image 1300 of a small animal eye showing anatomy and the relationship of Sagittal macro and micro structures. Special attention here showing specifically the morphology of the ciliary muscles and body.

FIG. 13B shows an example embodiment of a close-up cross-sectional 7T MRI image 1410 of a small animal eye SE showing whole eye anatomy and the relationship of Sagittal macro and micro structures. Special attention here showing specifically the morphology of the ciliary muscles and body. This is a zoomed in version of FIG. 13A.

FIG. 13C shows an example embodiment of a cross-sectional 7T MRI image 1320 of a small animal eye GE showing a whole eye ciliary body. FIGS. 13A-13C provide indications of ciliary muscles 1302.

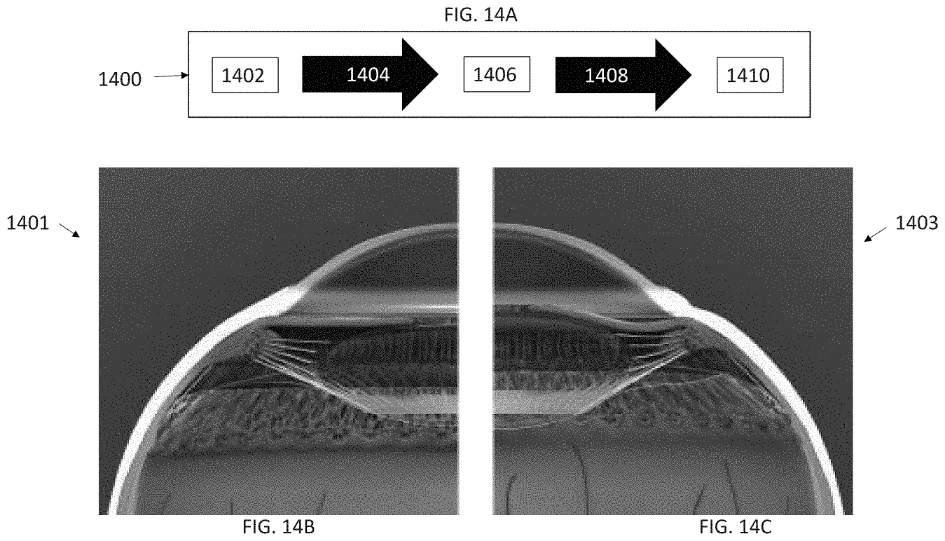

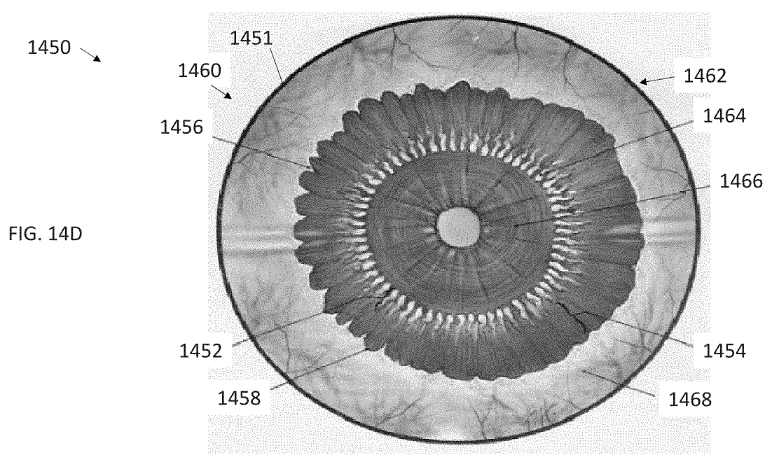

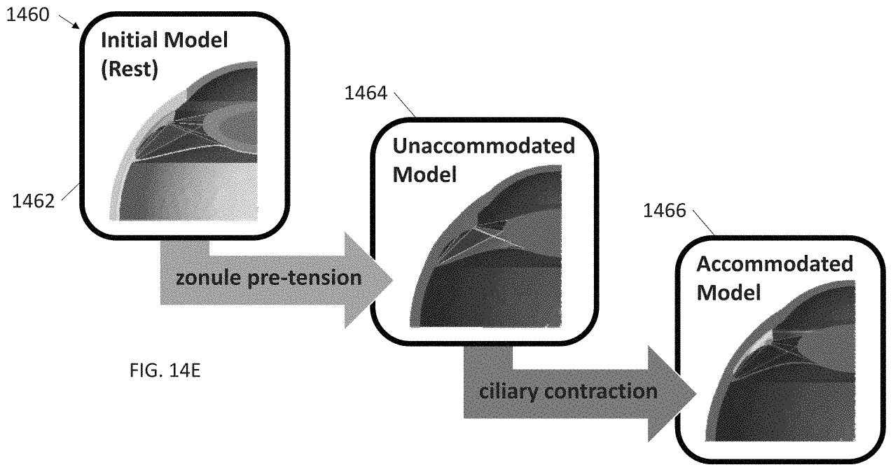

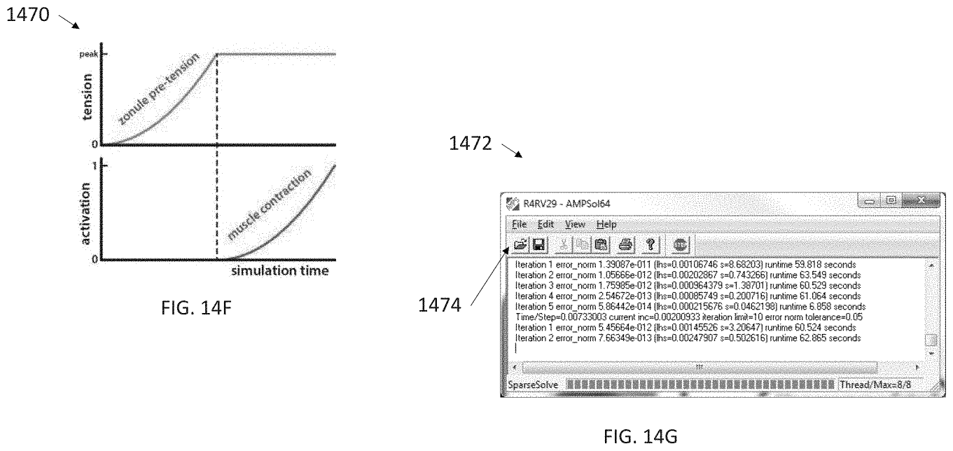

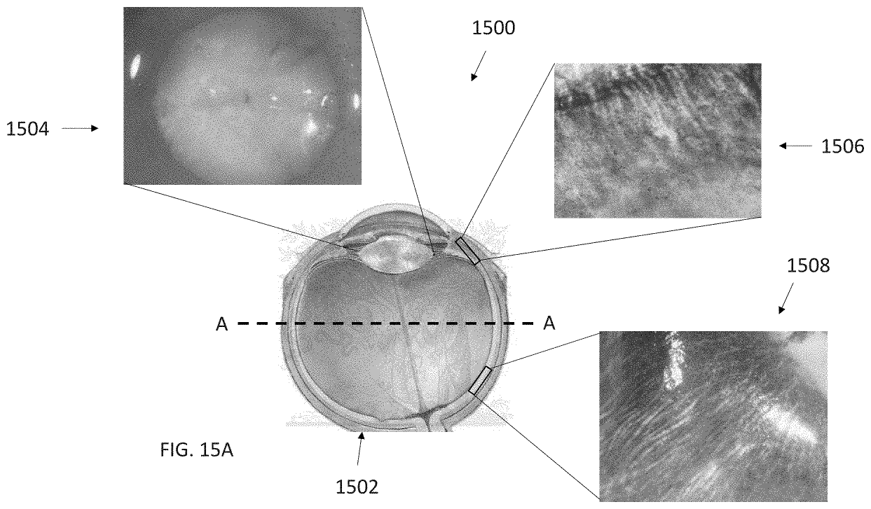

FIG. 14A shows an example embodiment of a simulation flowchart 1400 showing an initial model at rest undergoing zonule pre-tensioning to become an unaccommodated model and ciliary muscle contraction to become an accommodated model. As shown in the example embodiment, a two-dimensional or three-dimensional initial model 1402 has been developed and implemented in a computer. Initial model 1402 represents the eye at rest. As a first simulation step, conditions that represent a zonule pre-tensioning can be applied in step 1404. This zonule pre-tensioning will lead to the simulation modeling an unaccommodated eye model 1406. As described herein, unaccommodated eye model 1406 represents the eye when viewing things at a distance. Unaccommodated eye model 1406 can then be subjected to conditions that represent a ciliary muscle contraction in a second simulation step 1408. This ciliary muscle contraction simulation step 1408 will then cause the simulation to present an accommodated eye model 1410.

FIG. 14B shows an example embodiment of an unaccommodated eye diagram 1401.

FIG. 14C shows an example embodiment of an accommodated eye diagram 1403. FIGS. 14B-14C are shown side by side so that differences in ocular structures and positions can be seen in order to highlight their distinctions. These distinctions are discussed elsewhere herein.

FIG. 14D shows example embodiment diagram 1450 calling out various components of the anatomy of an eye 1451. As shown in the example embodiment, the pars plicata 1452 and pars plana 1454 are important ocular structures. A nasal side 1460 of the ocular structures includes a proceso dentado 1456, pars plicata 1452, and ora serrata 1458. A temporal side 1462 includes a proceso ciliar 1464 and pars plana 1454. An iris 1466 is centrally located and retina is located exteriorly.

FIG. 14E shows an example embodiment diagram 1460 of an accommodation simulation process. As shown in the example embodiment, an initial model 1462 can be a resting model. After simulating zonule pre-tentioning, an unaccommodated model 1464 can be created. Next, ciliary contraction can be simulated and an accommodated model 1466 can be created. This can be performed using AMPSol64 or other programs executed by a computer.

FIG. 14F shows an example embodiment diagram 1470 showing tension of zonules versus simulation time and ciliary muscle activation versus time.

FIG. 14G shows an example embodiment user interface diagram 1472 of an informational display during simulation screen. As shown in the example embodiment, the process can be tracking by iteration and timing, and information such as status and others can be displayed for the user. Users can save, open, print, copy, cut, and stop simulations from running by selecting the appropriate buttons 1474.

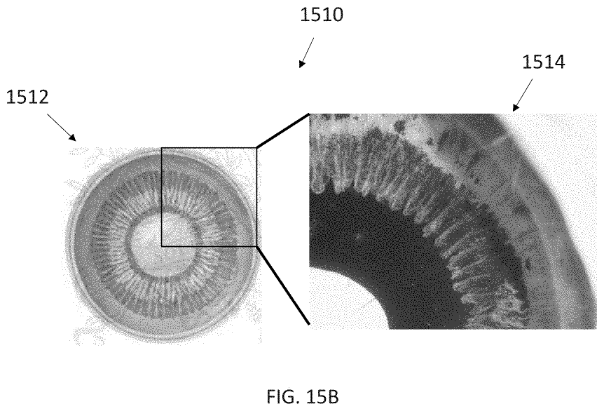

FIG. 15A shows an example embodiment of a diagram 1500 including a cross-sectional diagram 1502 of an eye with expanded lens image 1504, expanded ciliary muscle for confocal image 1506, and expanded choroid image 1508 taken using a bright scope across plane A-A.

FIG. 15B shows an example embodiment diagram 1510 including a cross-sectional diagram of an eye 1512 including a ciliary muscle and processes image 1514 taken using a bright scope.

FIGS. 16A-16C are cross-sectional confocal images 1600, 1602, 1604 respectively, showing ciliary fiber structures and fiber orientations. This data can be taken from cadaver eyes to determine fiber directions during movements. Here, eye imaging includes: Confocal Imaging of the 3 different fiber directions of the radial, longitudinal and circular muscles of the ciliary muscle or ciliary body. Each FIG. 16B is a zoomed version of FIG. 16A, and FIG. 16C is a further zoomed image that shows an example embodiment of an image of fiber orientation and branching.

FIG. 16D shows an example embodiment diagram 1610 of three parts of the ciliary muscle structure. The ciliary body 1612 contains the ciliary muscle. There are three types of muscle fibers: circular 1614, radial or oblique 1616, and longitudinal or meridonal 1618. Longitudinal muscle 1618 is also known as Bruke's muscle. The radial 1616 and longitudinal 1618 muscle fibers terminate in the scleral spur 1620. The longitudinal muscle fibers 1618 terminate in “epichoroidal stars” 1622 for attachment to the choroid layer at the ora serrata.

FIGS. 16E-16F show example embodiment diagrams 1630, 1650 of a corneo-scleral shell with a ciliary body. As shown in the example embodiment, sclera 1624 can be exterior to a choroid layer 1626. A transition from the choroid layer 1626 to the ciliary body 1612 is shown at the ora serrata 1628. Also shown is the cornea 1632.

FIG. 16G shows an example embodiment diagram 1660 of changes in the eye between an unaccommodated eye in central section 1662 for distance vision and accommodated eye in right section 1664 for near vision. As shown in the example embodiment, lens 1666 becomes thicker and more curved in accommodated vision and zonule fibers 1668 are under more tension.

FIGS. 16H-16I show example embodiments of a disaccomodated eye ciliary muscle diagram 1670 from a top view and accommodated eye ciliary muscle diagram 1672 from a top view, respectively. Muscle force during accommodation in shown by the arrows in FIG. 16I

FIGS. 16J-16K show example embodiments of a computer model of ciliary muscles of an eye from a top view 1674 and side cross-sectional view 1676 with inset respectively. As shown in the example embodiment, circular fibers 1614, radial fibers 1616, and longitudinal fibers 1618 can each be individually modeled.

FIGS. 16L-16N show example embodiment diagrams of longitudinal fibers 1678, radial fibers 1680, and circular fibers 1682, individually modeled and operable to be show simulations of their function during the accommodative process.

FIG. 160 shows an example embodiment diagram 1680 of normalized force versus relative length of ciliary muscle. This indicates that it is transversely isotropic, incompressible material with active contraction and three sets of fiber directions. Here, contraction is the force produced along muscle fibers. This indicates that ciliary muscle is best matched as “smooth striated” muscle.

Here, arrows indicate the contraction and movement of the ciliary body 1612. When the ciliary muscle 1612 contracts, the longitudinal fibers stretch choroid 1626 and pull ora serrata 1628 upwards toward cornea 1632. The end of the ciliary body 1612 close to the scleral spur 1620 is called the pars plicata. As the ciliary muscle 1612 contracts, the pars plicata moves inward and upward. This relaxes the tension on zonules attached to the crystalline lens, allowing the lens to take a steeper shape for near vision. As such, contraction of ciliary body 1612 stretches choroid 1626 and causes inward and upward movement of the pars plicata, relaxing zonules. Additionally, circular fibers 1614 have an increase in the cross-sectional size of their bundle.

The contraction of muscle is governed by protein interactions in the sub-units, called sarcomeres. When this contraction occurs, force is produced in the muscle in the direction of its fibers. The force produced is a function of the sarcomere length, where more force is produced at mid-length and much less is produced at the extremes of long and short. To model the forces in the ciliary muscle, assumptions about lengths during contraction are made based on previous research. Which direction the fibers are contracting to estimate the directions of the forces that the muscle produces are also important.

The longitudinal fibers run from the scleral spur to the ora serrata. The circular fibers run circumferentially around the lens. Between these are the intermediate fibers which transition between the two previous groups. Our model will include two muscle sections with longitudinal and circular fiber directions and a joined boundary between them. When the muscle fibers of the ciliary contract during accommodation, forces will be produced toward the center and front of the eye.

Muscle fiber arrangement and the directions of individual forces produced during accommodation can be used to specifically see their structure and function for each of the different fiber directions. To do this in the model fiber directions for the model must first be incorporated because the muscle forces flow through the fibers. Fiber direction determination is necessary in order to know the exact forces when simulated. A last step in setting up a model to accommodate through simulation. Thus, at this point all the things required for model creation are complete and ready for simulation, including the following: geometry, material properties, physics, fiber direction, and others.

Validation of the model can be performed by comparing measurements of known eye accommodation movements. In general, the lens may be simplified and move in a general way or be more specific. As such, adding a preload to the lens can assure that when the eye is unaccommodated the lens is stretched. Deformation in accommodation can also balance out the ratio of lens A/P movement and lens centripetal movement. Further refinement of lens movement with preloading can be performed and quantification and correlation of central optical power with lens movement as well. Once the accommodated-unaccommodated model is completed elastic forces and storage of energy potential can be measured and analyzed. This can allow for quantifying the potential energy stored in the choroid during stretching movements and also the longitudinal forces upon disaccommodation of the eye.

Validation of the modeling can occur by comparing results from the model with experimental data by different people and organization. This can allow for greater understanding of how the model operates and known ocular changes. Changes in both shape and position of ciliary muscles and the lens can be measured and compared with any measurements from imaging studies.

Comparison of model results may indicate that additional data needs to be collected since measured data is highly variable. Resolution and accuracy of the images themselves can be a cause of this variability. Thus, the question of “Is the model working as expected” can be answered yes, since it shows a similar trend even there is variability in the actual measured data.

Changes in ciliary ring lens equator diameters can also occur due to accommodation. Previously, measurements of the diameter of both the ciliary muscles and the lens have been performed on unaccommodated and accommodated eyes. This data is shown by two lines. In the figure. Unaccommodated points on the left figure and accommodated points are shown on the right figure. These were measured over a range. Previously, it was reported that there was no real correlation between ciliary ring diameter and optical power. Validation of the model using trends has been shown to match this data.

Changes in lens forward A/P displacement with accommodation has been shown to match the model as well, as shown in the figure. Further, changes in lens thickness with accommodation can be validated. Here, even though the model is right at the median or average of the data, not much thickening of the lens is shown. Thus, it appears too flat. However, this can be explained by the forces of the prestressing.

Refining the model can be performed by modifying lens movement by adding the pre-stress, performing ciliary muscle fiber studies using 3D imaging (such as by imaging cadaver eyes), and by adding the Limbal ring. Further, model parameters can be varied to investigate measured physiologic changes associated with presbyopia. Additionally, utility of the model can be demonstrated by examining the effect of surgical corrections to presbyopia. Since the model demonstrates accommodation of a young healthy eye, varying the model can demonstrate accommodation in presbyopic eyes.

FIG. 16P shows an example embodiment chart 1682 of force versus muscle length, indicating that the top of the pyramidal shape could be the “sweet spot.”

FIG. 16Q shows an example embodiment of a disaccomodated eye diagram 1684 and accommodated eye diagram 1686. Here, a scleral spur 1688 is shown at the top of the figure. When accommodation occurs, meridional muscle 1690 contracts, ora serrata 1692 is pulled up and retina/choroid 1694 stretches with respect to sclera 1696 due to a weak shearing.

FIG. 16R shows an example embodiment diagram 1698 of a simple spring model of ciliary muscle movement. Here, average radial choroid modulus can be about 8×10⁵ N m⁻² (0.8 MPa), while average radial sclera modulus can be about 2×10⁶ N m⁻² (2.0 MPa).

FIG. 17A shows an example embodiment screenshot 1700 of a model of ocular structures for use in simulation. As shown, ciliary muscle 1702 movement can be simulated by inputting initial conditions and running simulations, such as during an accommodative process, along with other ocular structural movement. Thickness changes are shown by the arrows.

FIG. 17B shows an example embodiment image 1708 of individual ciliary fiber movement during an accommodative process including thickness changes, as indicated by the arrows.

FIG. 17C shows an example embodiment image 1706 indicating overall ciliary muscle movement during an accommodative process including changes in thickness, as indicated by the arrows.

FIG. 17D shows an example embodiment diagram 1708 of ciliary muscle thickness at ciliary muscle apex versus accommodative amount. As shown in the example embodiment, a simulation was run using finite element modeling, as shown by the line. Various individual data points from clinical studies performed previously are also mapped, indicating that the model and simulator effectively shows the thickness changes measured.

FIG. 17E shows an example embodiment screenshot 1710 of a model of ocular structures for use in simulation. As shown in the example embodiment, diameters of ciliary body 1702 and lens 1712 can be measured and simulated according to the model.

FIG. 17F shows an example embodiment image 1714 of ciliary muscle and lens movement during an accommodative process including diameter changes, as indicated by the arrows.

FIG. 17G shows an example embodiment diagram 1716 of ciliary muscle ring diameter versus accommodative amount. As shown in the example embodiment, a simulation was run using finite element modeling, as shown by the line. Various individual data points from clinical studies performed previously are also mapped, indicating that the model and simulator effectively shows the diameter changes measured.

FIG. 17H shows an example embodiment diagram 1718 of lens diameter versus accommodative amount. As shown in the example embodiment, a simulation was run using finite element modeling, as shown by the line. Various individual data points from clinical studies performed previously are also mapped, indicating that the model and simulator effectively shows the lens diameter changes measured.

FIG. 17I shows an example embodiment screenshot 1720 of a model of ocular structures for use in simulation. As shown in the example embodiment, forward displacement of lens 1712 can be measured and simulated according to the model.

FIG. 17J shows an example embodiment image 1722 of forward displacement of lens during an accommodative process, as indicated by arrow 1724. Other arrows show changes in other ocular structures.

FIG. 17K shows an example embodiment diagram 1726 of forward displacement of the lens versus accommodative amount. As shown in the example embodiment, a simulation was run using finite element modeling, as shown by the line. Various individual data points from clinical studies performed previously are also mapped, indicating that the model and simulator effectively shows the forward displacement of the lens during accommodation.

FIG. 17L shows an example embodiment screenshot 1728 of a model of ocular structures for use in simulation. As shown in the example embodiment, changes in thickness of lens 1712 can be measured and simulated according to the model.

FIG. 17M-17N show example embodiment images 1730, 1732 of lens thickness changes during an accommodative process, as indicated by the arrows.

FIG. 17O shows an example embodiment diagram 1734 of lens thickness changes versus accommodative amount. As shown in the example embodiment, a simulation was run using finite element modeling, as shown by the line. Various individual data points from clinical studies performed previously are also mapped, indicating that the model and simulator effectively shows lens thickness changes during accommodation.

FIGS. 17P-17Q show example embodiment screenshots of an accommodated eye 1736 and unaccommodated eye 1738 model of ocular structures for use in simulation, respectively. As shown in the example embodiment, changes in ciliary muscle 1702 and lens 1712 can be measured and simulated according to the model. Here, lens 1712 can gain thickness and ciliary muscle 1702 can change position during accommodation.

FIGS. 17R-17S show example embodiment diagrams 1740, 1744 of changes to ciliary muscle 1742 and lens 1744 respectively, before, midway, and after an accommodative process. The solid lines indicate an unaccommodated shape, the medium dashed lines indicate midway accommodated, and the dark dashed lines indicate full accommodative shape.

FIG. 17T shows an example embodiment of a user interface diagram 1748 displaying measured results of positioning information during a simulation. As shown in the example embodiment, users can select particular features to follow or select positions of particular features during a simulation. Coordinates and distances between points or changes in position can be entered and displayed in various embodiments.

FIG. 18A shows an example embodiment of a 3-dimensional cross-sectional model structure diagram 1800 showing pre-tensioning of zonules 1818 and changes in the lens 1822 and ciliary body 1808 of an eye. As shown in the example embodiment, during modeling zonules 1818 can be pre-tensioned to change lens 1818 from normal or otherwise unaltered anatomic measurements of a resting shape to those of an unaccommodated shape. As such, lens 1822 becomes thinner and wider as a result of zonules 1818 pulling outward and downward into the eye, while fibers of ciliary body 1808 shorten to tension. Pre-tensioning of zonules 1822 prior to muscle contraction may be applied in order for a model to produce appropriate lens 1818 deformation. After applying the simulation to the model, results of displacement and deformation of lens 1822 and ciliary muscle 1808 can fall within the range of known values for accommodation of a young adult human eye, as described in existing medical literature and shown in FIG. 18B.

FIG. 18B shows an example embodiment of a chart 1850 showing accommodation of model results as a line using a 3-dimensional cross-sectional model, as compared with a prior art model that captured data points. Chart 1850 shows distance along fiber stretch in zonules versus lens thickness in millimeters. As shown, accuracy of three-dimensional modeling can be proven to be comparable an effective modeling technique compared with known data that exists in current medical literature. As described herein, systems, methods and devices including the pretensioning of ocular zonules conducts an instruction to modeling that elicits novel exploitation of biomechanical relationships and functions of the extra-lenticular structures of the eye as it relates to the mechanisms of accommodation and COP.

FIG. 19A shows an example embodiment of a 3-dimensional cross-sectional model structure diagram 1900 showing simulated accommodation of an eye through ciliary muscle 1908 contracting with varied muscle activation. As shown in the example embodiment, anterior and central contraction of ciliary muscles 1908 can be used to simulate accommodation of the eye. As such, this contraction causes lens 1922 to become thicker and more curved, as well as to shift in an anterior direction. However, it is known that ciliary muscles include sets of fibers, such as longitudinal fibers, radial fibers, and circular fibers. These fibers are known to function differently and produce different results, such that the contraction of specific fiber groups within ciliary muscle 1908 can contribute disproportionately to different aspects of lens 1918 shape-change during accommodation. FIGS. 19B-19D model each of these fiber groups independently.

FIG. 19B shows an example embodiment of 3-dimensional cross-sectional model structure diagram 1930 showing simulated accommodation of an eye through longitudinal ciliary fiber contraction and its associated muscle fiber trajectories. Further description of longitudinal ciliary fibers is shown and given with respect to FIGS. 9A-9B. As shown in the example embodiment, longitudinal fibers 1908a may be generally located on an exterior of ciliary muscle 1908. Thus, when longitudinal fibers 1908a are activated, the outer portions of ciliary muscle 1908 move. This is a movement with a shallow slope, compared to other fibers, as shown in muscle trajectory depiction 1962.

FIG. 19C shows an example embodiment of 3-dimensional cross-sectional model structure diagram showing simulated accommodation of an eye through ciliary contraction with varied muscle activation, particularly showing muscle fiber trajectories for radial fibers. Further description of radial ciliary fibers is shown and given with respect to FIGS. 9A-9D. As shown in the example embodiment, radial fibers 1908b may be generally located in a central or internal portion of ciliary muscle 1908. Thus, when radial fibers 1908b are activated, central or internal portions of ciliary muscle 1908 move. This is a movement with a steeper slope, compared to other fibers, as shown in muscle trajectory depiction 1964.

FIG. 19D shows an example embodiment of 3-dimensional cross-sectional model structure diagram showing simulated accommodation of an eye through ciliary contraction with varied muscle activation, particularly showing muscle fiber trajectories for circular fibers. Further description of circular ciliary fibers is shown and given with respect to FIGS. 9A-9D. As shown in the example embodiment, circular fibers 1908c may be generally located on an interior of ciliary muscle 1908. Thus, when circular fibers 1908c are activated, the inner portions of ciliary muscle 1908 move. This is a small movement, compared to other fibers, as shown in muscle trajectory depiction 1966.

For example, contraction of radial ciliary fibers 1908b can significantly contribute to anterior displacement of the lens, as shown in FIG. 19C. Contraction of circular ciliary fibers 1908c can contribute most significantly to thickening of the ciliary muscle at or near the apex, as shown in FIG. 19D, which can result in lens thickening and increased lens curvature.

FIG. 20A shows an example embodiment of a chart 2000 showing accommodation of model results using a 3-dimensional cross-sectional model structure diagram showing as compared with a prior art model for anterior displacement of a lens in millimeters. As shown in the example embodiment, the prior measurements were unable to determine which fibers were moving, and where. However, the three-dimensional simulation was able to monitor function of all fibers active, represented by line 2008; longitudinal fibers, represented by line 2008a, radial fibers, represented by line 2008b; and circular fibers, represented by 2008c. This is a vast improvement over the prior art.

FIG. 20B shows an example embodiment of a chart 2050 showing accommodation of model results using a 3-dimensional cross-sectional model structure diagram showing as compared with a prior art model for apex thickness of ciliary muscle in millimeters. As shown in the example embodiment, the prior measurements were unable to determine which fibers were moving, and where. However, the three-dimensional simulation was able to monitor function of all fibers active, represented by line 2058; longitudinal fibers, represented by line 2058a, radial fibers, represented by line 2058b; and circular fibers, represented by 2058c. This is a vast improvement over the prior art.

FIG. 21 shows an example embodiment of a cross-sectional ocular structure diagram 2160 showing ocular structures of a human eye. As shown in the example embodiment, an intra stromal disk implant 2162 can be placed within layers of a corneal stroma 2164 of cornea 2166. Cornea 2166 is coupled with limbus 2168 and canal of Schlemm 2170 is located posteriorly in cornea 2166. Fin 2172 is located anteriorly in bleb 2174 and sub-tenon SIBS disk implants 2176 can be placed posteriorly. Tenons 2178 can be located exterior to bleb 2174 and covered by conjunctiva 2180. MIDI Tube 2182 can be located between bleb 2174 and sclera 2184, which is located exterior to retina 2186. Ciliary muscles 2188 are coupled with ciliary body 2190, which are in turn coupled with ligaments of zonules 2192. Trabecular network 2194 is coupled with iris 2196, in turn covering a portion of lens 2198.

FIG. 22A shows an example embodiment diagram 2200 of treatment regions from a particular three zone model protocol. As shown, an inner zone1 2202, middle zone2 2204 and outer zone3 2206 can be circumferentially located about a central axis.

FIG. 22B shows an example embodiment diagram 2210 of treatment regions from a particular three zone model protocol. As shown, an inner zone1 2202 is shown individually in the upper left quadrant, middle zone2 2204 is shown in the upper right quadrant, outer zone3 is shown in the lower right quadrant, and composite of all three zones 2208 is shown in the lower left quadrant.

FIG. 22C shows an example embodiment diagram 2212 of a simulated medical treatment of an eye. As shown in the example embodiment, treatment to achieve a desired effect can be simulated using an eye model 2216. Here, a laser 2214 is generating a beam of energy for application at location 2220 on a sclera 2218 of eye model 2216. This simulation can be used to determine potential effects of treatment on an eye, for instance to help treat accommodative problems due to aging.

FIG. 22D shows an example embodiment diagram 2230 of a simulated medical treatment of an eye, including treatment regions from a particular three zone model protocol. As shown, an inner zone1 2202, middle zone2 2204 and outer zone3 2206 can be circumferentially located about a central axis at the right of the figure. These zones are shown as sections of sclera 2218.

FIG. 22E shows an example embodiment diagram 2232 of a simulated medical treatment of an eye, including treatment regions from a particular three zone model protocol. As shown, an inner zone1 2202, middle zone2 2204 and outer zone3 2206 can be circumferentially located about a central axis at the right of the figure. These zones are shown as sections of sclera 2218. Here, treatment of sclera 2218 can affect the movement of ciliary body by applying a laser to it. This beam may remove parts, portions, or sections of tissue, thus changing the biomechanical properties of the underlying ciliary muscle 2234. This can affect the length and apex thickness of the ciliary muscle during an accommodative process.

FIG. 22F shows an example embodiment chart 2236 of macro results of therapy simulation methods. As shown in the example embodiment, a baseline simulation can include a first accommodation model with an “old” sclera. An initial presumption is that age-related changes that contribute to presbyopia cause various effects. For example, the eye lens may become more stiff, the ciliary body may be impeded by stiffening of its posterior attachments, the ciliary muscle may lose contractility, and the lens itself may grow, which can lead to reduced tension in the zonules when at rest. Therefore, when creating a simulation, previous computational models can be applied to assess the individual effects of various structures on accommodative function. These changes can be applied in isolation using these new simulations by applying individual changes to various factors. These can include the following: lens stiffness, sclera stiffness, the sclera attachment to the ciliary muscles and choroid, which can also be coupled with stiffness changes, zonular tension changes, ciliary muscle contraction, and others. In various embodiments, it is beneficial to run simulations with changes from the eye of a thirty-year-old individual to that of a seventy-year-old individual. These simulations can be used to determine which structural changes cause the greatest effects and can highlight the most likely mechanisms of presbyopia. As such, ideal candidates for actual treatments can be identified based on the influence of different changes by simulated age.

Here, a stiff sclera can be set with a modulus of elasticity (E)=2.85 MPa, equivalent to that of an individual of about 50 years old. A tight attachment between the sclera and the ciliary body and choroid can occur and all other parameters can be changed. These include ciliary activation, stiffness of other components, and others as appropriate.

Next, treatment simulations can include use of the baseline model with regionally “restored” sclera stiffness and attachment tightness. This can simulate treated combinations of changes to different zones, both with and without changing attachment by modifying parameters. These changes can be performed in zones: 1, 2, 3, 1+2, 2+3, 1+2+3, and others. As such, a restored sclera can have a modulus of elasticity (E)=1.61 MPa, equivalent to an individual of about 30 years old. These values can simulate a loose attachment between sclera and the ciliary body and choroid. An effect of regional treatment on ciliary deformation in accommodation can be seen in FIGS. 22G-22H, including apex thickening and length shortening, both in millimeters, as shown.

FIG. 22G shows an example embodiment chart 2238 of apex thickness of the ciliary body for various zones simulated, along with a baseline. Here, better results are shown by higher locations on the chart.

FIG. 22H shows an example embodiment chart 2240 of length shortening of the ciliary body for various zones simulated, along with a baseline. Here, better results are shown by higher locations on the chart.

FIG. 22I shows an example embodiment chart 2242 of micro results for therapy simulation methods. Here, pores are made in tissue that can affect biomechanics in the tissue and surrounding or coupled tissues. As shown in the example embodiment, a restored sclera stiffness can be dependent on the treatment, based on the density of pores. Pore density can be a factor of the percent volume of material removed and can be varied by changing parameters of these pore ablation holes. Parameters can include depth, diameter, quantity, and others as appropriate. Therefore, the resultant stiffness is estimated as a microscale mixture of holes and is assumed to be parallel or evenly spaced and sized with volume equals treatment density or percent of the total. The remaining volume is “old” sclera (E=2.85 MPa). In some embodiments, it has been shown in simulation that remove of about 43.5% of volume operates to change sclera stiffness from older, about 50 years old, to younger, about 30 years old.

FIG. 22J shows an example embodiment diagram 2244 of different characteristics of pore density that can be changed. First is depth 2246, pore width 2248, and quantity 2250.

As a result of these simulations, various questions can be answered by using the model, as follows: First, how does regional restoration of sclera stiffness improve ciliary deformation in accommodation and do certain zones or combinations of zones have a greater effect? Here, treating all 3 zones resulted in the most improved deformation at the ciliary's length and apex; individually treating zone 2 had the greatest effect, while treating zone 3 had the least.

Second, does regional restoration of sclera attachment tightness, in addition to stiffness, augment improvements to ciliary deformation in accommodation? Here, treatment in zones 2 and 3 had a much greater affect in improving ciliary deformation at the apex, corresponding with increasing lens thickness, if the attachment of the sclera to the ciliary/choroid was assumed to return too loose instead of tight.

Third, how do the treatment parameters relate to the change in scleral stiffness in the treated regions? Here, sclera stiffness decreases linearly with increasing treatment density, by the amount of volume removed, that can be determined by the hole diameter and depth as well as the total number of holes. Thus 43% of the volume needs to be removed achieve the same stiffness as the sclera in the accommodating model of an individual about 30 years old.

Fourth, how does regional restoration with different treatments, including different sclera stiffness's, improve ciliary deformation in accommodation? Here, treatments with increasing density improve ciliary deformation at the apex and length. However, changing the stiffness has a limited affect without also changing the attachment tightness.

Additional questions that may be answered with further experimentation include the following: does sclera's attachment to ciliary become tighter with age, do procedures alter the tightness of this attachment in addition to changing regional sclera stiffness, and others.

FIG. 23 shows an example embodiment diagram 2300 of treated stiffness including modulus of elasticity of sclera in a treated region versus volume fraction or percent of sclera volume removed in the treated region for the simulation.

FIG. 24A shows an example embodiment diagram 2251 of a simulated medical treatment of an eye, including treatment regions from a particular five zone model protocol. As shown, an inner zone0 2252, second inner zone1 2202, zone2 2204 and outer zone3 2206, and additional outer zone 2256 can be circumferentially located about a central axis at the right of the figure. These zones are shown as sections of sclera 2218. Here, treatment of sclera 2218 can affect the movement of ciliary body by applying a laser to it. This beam may remove parts, portions, or sections of tissue, thus changing the biomechanical properties of the underlying ciliary muscle 2234. This can affect the length and apex thickness of the ciliary muscle during an accommodative process.

FIG. 24B shows an example embodiment chart 2260 of macro results of therapy simulation methods. In the example embodiment, baseline simulation: original model of healthy accommodation with “old” sclera with a stiff sclera: modulus of elasticity (E)=2.85 MPa, equivalent to about a 50-year-old's eye. This can have a tight attachment between the sclera and the ciliary/choroid. All other parameters changed, including ciliary activation, stiffness of other components, and others. Treatment simulations include a baseline model with regionally “treated” sclera stiffness and attachment tightness. These can include treated combinations of zones (with & without changing attachments individually for zones: 0, 1, 2, 3, 4; combined: 1+2+3, 1+2+3+4, 0+1+2+3+4. The treated sclera can have a modulus of elasticity (E)=1.61 MPa, equivalent to that of about a 30-year-old's eye. This eye has a loose attachment between the sclera and the ciliary/choroid and values in an original accommodation model.

FIG. 24C shows an example embodiment chart 2262 of apex thickness of the ciliary body for various zones simulated, along with a baseline, and results that affect scleral stiffness only. Here, better results are shown by higher locations on the chart.

FIG. 24D shows an example embodiment chart 2264 of length shortening of the ciliary body for various zones simulated, along with a baseline, and results that affect scleral stiffness only. Here, better results are shown by higher locations on the chart.

FIG. 24E shows an example embodiment chart 2266 of macro results of therapy simulation methods and results that affect scleral stiffness and attachment.

FIG. 24F shows an example embodiment chart 2268 of apex thickness of the ciliary body for various zones simulated, along with a baseline, and results that affect scleral stiffness and attachment. Here, better results are shown by higher locations on the chart.

FIG. 24G shows an example embodiment chart 2270 of length shortening of the ciliary body for various zones simulated, along with a baseline, and results that affect scleral stiffness and attachment. Here, better results are shown by higher locations on the chart.

FIG. 24H shows an example embodiment chart 2400 of effects of treatment density on ciliary deformation in accommodation that affect scleral stiffness only. Here, sclera in all zones changed to stiffness corresponding with volume fraction of treatment for tight attachment. Treatment stiffness=(1−(volume fraction)/100)×baseline stiffness.

FIG. 24I shows an example embodiment chart 2402 of apex thickness of the ciliary body for various zones simulated versus volume faction percent removed. A protocol range is shown as well as decreased scleral thickness and “young” stiffness.

FIG. 24J shows an example embodiment chart 2404 of length shortening of the ciliary body for various zones simulated versus volume faction percent removed. A protocol range is shown as well as decreased scleral thickness and “young” stiffness.

FIG. 24K shows an example embodiment chart 2406 of effects of treatment density on ciliary deformation in accommodation that affect scleral stiffness and attachment. Here, sclera in all zones changed to stiffness corresponding with volume fraction of treatment for tight attachment.

FIG. 24L shows an example embodiment chart 2408 of apex thickness of the ciliary body for various zones simulated versus volume faction percent removed. A protocol range is shown as well as decreased scleral thickness and “young” stiffness and healthy apex thickening line reference. These results are shown for, tight attachments, loose attachments and changing attachments.

FIG. 24M shows an example embodiment chart 2410 of length shortening of the ciliary body for various zones simulated versus volume faction percent removed. A protocol range is shown as well as decreased scleral thickness and “young” stiffness and healthy length shortening line reference. These results are shown for, tight attachments, loose attachments and changing attachments.

Here, the “treated” sclera stiffness is dependent on volume fraction percent sclera volume removed by treatment. The resultant stiffness estimated as microscale mixture of holes that are assumed to be parallel evenly spaced, sized within a volume that equals the volume fraction or is a percentage of total sclera volume. As such, any remaining volume is “old” sclera (E=2.85 MPa). It was found that there is a need to remove about 43.5% of volume to change sclera stiffness from old a fifty-year old simulated eye to receive the benefits of having a younger thirty-year-old eye. Protocols or combinations of density percentage and depth allow for a maximum volume fraction of 13.7 percent, equivalent to a new stiffness of 2.46 MPa. It should be understood that different numbers of zones and pores can be used in different treatment methods.

Next, does regional restoration of sclera attachment tightness and stiffness augment improvements to ciliary deformation in accommodation? Here, individually treating zones 1 & 2 had a much greater affect in improving ciliary deformation at the apex (corresponding with increasing lens thickness) if the attachment of the sclera to the ciliary/choroid was assumed to return too loose instead of tight. Simultaneously treating zones 1-4 (+/−zone 0) had a very large effect on deformation of both ciliary length and apex.

Further, how do the treatment parameters relate to the change in scleral stiffness in the treated regions? Here, scleral stiffness decreases linearly with increasing volume fraction of the amount of volume removed that can be determined by the pore density percentage as a function of the spot size and number of pores, and depth. This resulted in 43% of the volume needs to be removed achieve the same stiffness as the sclera in the accommodating model of about a 30-year-old's eye.

Additionally, how does regional restoration with different treatments (therefore different sclera stiffness's) improve ciliary deformation in accommodation? Here, treatments with increasing density improved ciliary deformation at the apex and length. However, changing the stiffness has a limited affect without also changing the attachment tightness.

Algorithms and other software used to implement the systems and methods disclosed herein are generally stored in non-transitory computer readable memory and generally contain instructions that, when executed by one or more processors or processing systems coupled therewith, perform steps to carry out the subject matter described herein. Implementation of the imaging, modeling and other subject matter described previously can be used with current and future developed medical systems and devices to provide benefits that are, to date, unknown in the art.

FIG. 25A is an example embodiment of a basic network setup diagram 2500. As shown in the example embodiment, network setup diagram 2500 of can include multiple servers 2540, 2550 which can include applications distributed on one or more physical servers, each having one or more processors, memory banks, operating systems, input/output interfaces, power supplies, network interfaces, and other components and modules implemented in hardware, software or combinations thereof as are known in the art. These servers can be communicatively coupled with a wired, wireless, or combination network 2510 such as a public network (e.g. the Internet, cellular-based wireless network, cloud-based network, or other public network), a private network or combinations thereof as are understood in the art. Servers 2540, 2550 can be operable to interface with websites, webpages, web applications, social media platforms, advertising platforms, and others. As shown, a plurality of end user devices 2520, 2530 can also be coupled to the network and can include, for example: user mobile devices such as smart phones, tablets, phablets, handheld video game consoles, media players, laptops; wearable devices such as smartwatches, smart bracelets, smart glasses or others; and other user devices such as desktop devices, fixed location computing devices, video game consoles or other devices with computing capability and network interfaces and operable to communicatively couple with network 2510.

FIG. 25B is an example embodiment of a network connected modeling and simulation system diagram 2540. As shown in the example embodiment, a modeling and simulation server system can include at least one user device interface 2547 implemented with technology known in the art for facilitating communication between system user devices and the server and communicatively coupled with a server-based application program interface (API) 2550. API 2550 of the server system can also be communicatively coupled to at least one tracking and routing engine 2548 for communication with web applications, websites, webpages, websites, social media platforms, and others. As such, it can access information via a network when needed. API 2550 can also be communicatively coupled with a parameter database 2542, a historical research informational database 2543, a mathematical model database 2545, and results database 2546 combinations thereof or other databases and other interfaces. API 2550 can instruct databases 2542, 2543, 2545, 2546 to store (and retrieve from the databases) information such as variables, models, best practices, results, or others as appropriate. Databases 2542, 2543, 2545, 2546 can be implemented with technology known in the art, such as relational databases, object-oriented databases, combinations thereof or others. Databases 2542, 2543, 2545, 2546 can be a distributed database and individual modules or types of data in the database can be separated virtually or physically in various embodiments.

FIG. 25C is an example embodiment of a user mobile device diagram 2521. As shown in the example embodiment, a user mobile device 2521, can includes a network connected simulation application 2522 that is installed in, pushed to, or downloaded to the user mobile device or its internet browser application. In many embodiments user devices are touch screen devices such as smart phones, phablets or tablets which have at least one processor, network interface, camera, power source, memory, speaker, microphone, input/output interfaces, operating systems and other typical components and functionality. It should be understood that user mobile device 2521 can be replaced with equivalent functionality by user devices such as desktop or laptop computers in various embodiments.

In some embodiments, simulation application 2522 may not be installed on user device 2521. Instead, it may be replaced by one or more of a system administrator application, an advertiser application, an affiliate application, a consumer application, or others. In some embodiments, a dedicated application for any of these may not be installed on user device 2521. Instead, users may access a portal via a web browser installed on device 2521, which may be dedicated or hybrids of various portals or websites.

Although FIGS. 25A-25C are directed to a network-based system, it should be understood that simulations and modeling systems and processes and data storage in non-transitory memory as disclosed herein can be performed on non-network connected devices as well. Further, in some embodiments, they are distributed in different fashions than those shown.

As used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances, entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. A computer-implemented method of three-dimensional modeling for the treatment of accommodation of an eye, the method comprising:
determining, using a processor, a first anatomic model of one or more structures of the accommodative mechanism of the eye of a patient, wherein the one or more structures associate with at least one of ciliary muscle, lens, zonules, sclera, and choroid:
determining a three-dimensional biomechanical model of the one or more structures of the eye using at least the first anatomic model;
determining one or more parameters associated with a changed biomechanical state of the eye and related crystalline lens, wherein the one or more parameters include at least one of scleral stiffness and lens stiffness; and
determining a second anatomic model incorporating geometric changes to the first anatomic model in response to the changed physiological state, using the three-dimensional biomechanical model and the one or more parameters associated with the changed biomechanical state.

2. The method of claim 1, wherein the biomechanical state includes a baseline state, an age-related physiological state, a biomechanical functional state, and a biomechanical dysfunctional state.

3. The method of claim 1, wherein the one or more parameters are associated with biomechanical conditions optical conditions, boundary conditions, or a combination thereof.

4. The method of claim 1, further comprising:
performing a simulation using the biomechanical model, wherein the one or more parameters associated with the changed biomechanical state of the patient are determined using the simulation.

5. The method of claim 4, wherein the simulation includes a simulation of accommodation of the eye.

6. The method of claim 1, further comprising:
selecting one or more portions of the first anatomic model, wherein the biomechanical model includes a model of one of the one or more portions of the first anatomic model.

7. The method of claim 1, wherein the biomechanical model includes at least one of measurements or properties of a scleral wall and choroid.

8. The method of claim 1, further comprising:
performing a simulation using the second anatomic model; and
outputting results of the simulation.

9-21. (canceled)
